SEQUENCE LISTING (2 sequences)

SEQ ID NO: 1 - DNA, 18 bp, Artificial Sequence
  Oligonucleotide primer
gctgcctcccgtaggagt

SEQ ID NO: 2 - DNA, 18 bp, Artificial Sequence
  Oligonucleotide primer
actcctacgggaggcagc

(19) United States
(12) Patent Application Publication
(10) Pub. No.: US 2022/0023419 A1
(43) Pub. Date: Jan. 27, 2022

(54) INHIBITING INFLAMMATION

(71) Applicant: THE COUNCIL OF THE QUEENSLAND INSTITUTE OF MEDICAL RESEARCH, Queensland (AU)

(72) Inventors: Geoffrey Hill, Seattle, WA (US); Motoko Koyama, Seattle, WA (US)

(21) Appl. No.: 17/298,312

(22) PCT Filed: Nov. 28, 2019
(86) PCT No.: PCT/AU2019/051306

§ 371 Date: May 28, 2021

(30) Foreign Application Priority Data
Nov. 28, 2018 (AU) 2018904524

(51) Int. Cl. A61K 39/395; A61K 38/14; A61K 38/12; A61K 31/7048; A61K 31/7036; A61K 31/546; A61K 35/741; A61K 35/24; A61K 38/08; A61P 37/06

(52) U.S. Cl. CPC A61K 39/3955; A61K 38/14; A61K 38/12; A61K 31/7048; A61P 37/06; A61K 31/546; A61K 35/741; A61K 35/24; A61K 38/08; A61K 31/7036

(57) ABSTRACT

The present disclosure relates to methods of reducing inflammation in the gastro-intestinal (GI) tract, and more specifically to methods of reducing the risk of transplant recipients developing graft-versus-host disease by reducing inflammation in the gut.

### CROSS-REFERENCE TO RELATED APPLICATIONS

The present application claims priority from Australian Provisional Patent Application No 2018904524 filed on 28 Nov. 2018, the content of which is incorporated herein by reference.

### TECHNICAL FIELD

The present disclosure relates to methods of reducing inflammation in the gastro-intestinal (GI) tract, and more specifically to methods of reducing the risk of transplant recipients developing graft-versus-host disease by reducing inflammation in the gut.

### BACKGROUND

The principal function of the immune system is to respond to pathogens in a timely and appropriate manner. This requires a delicate balance of tightly regulated responses, especially at barrier sites, such as the skin and the GI tract, which are continuously exposed to microbial and environmental challenges. Antigens are presented to T cell receptors (TCR) on CD4⁺ and CD8⁺ T cells by major histocompatibility complex (MHC) class-II and class I, respectively. Current dogma suggests that MHC class-II-dependent immunity is initiated by “professional” hematopoietic-derived antigen presenting cells (APC) including dendritic cells (DC), macrophages, monocytes and B cells. While non-hematopoietic cells including mesenchymal cells and epithelial cells can express MHC class-II, its physiological and pathological significance in in vivo settings remains to be elucidated. The nature and relative importance of the APC that operate in the GI tract under physiological and inflammatory conditions remain unknown.

The GI tract plays a critical role in many inflammatory conditions including graft-versus-host disease (GVHD) following allogeneic bone marrow transplantation (BMT). Acute GVHD is the often fatal manifestation of immunopathology mediated by donor T cells in response to alloantigen presented by an undefined APC. Importantly, acute GVHD of the GI tract is the prima facie determinant of disease severity and lethality. Damage to the GI tract plays a major role in the initiation and amplification of systemic inflammation and subsequent GVHD and fatal GVHD is almost always a consequence of GI tract involvement.

Accordingly, there remains a need for methods to reduce inflammation in the gastrointestinal track and to reduce the risk of GVHD in transplant recipients.

### SUMMARY

The present disclosure relates to the finding that the innate and adaptive immune responses to the microbiome play a role in shaping antigen presentation by intestinal epithelial cells, and that when these responses are dysregulated, inflammation and tissue pathology ensues.

Accordingly, the present disclosure provides a method of reducing the risk of a transplant recipient developing graft versus host disease (GVHD), the method comprising administering a composition to the transplant recipient to modulate the transplant recipient's gut microbiome, and/or administering a composition to reduce MHC class II expression on intestinal epithelial cells (IEC) in the transplant recipient, wherein the composition is administered prior to the transplant recipient receiving pre-transplant conditioning therapy.

In one embodiment, the method comprises administering the composition to modulate the transplant recipient's gut microbiome at least three days prior to the transplant recipient receiving the pre-transplant conditioning therapy, and/or administering the composition to reduce MHC class II expression on IEC in the transplant recipient at least three days prior to the transplant recipient receiving the pre-transplant conditioning therapy.

In another embodiment, the method comprises administering the composition to modulate the transplant recipient's gut microbiome at least seven days prior to the transplant recipient receiving the pre-transplant conditioning therapy, and/or administering the composition to reduce MHC class II expression on IEC in the transplant recipient at least seven days prior to the transplant recipient receiving the pre-transplant conditioning therapy.

In yet another embodiment, the method comprises administering the composition to modulate the transplant recipient's gut microbiome at least two weeks prior to the transplant recipient receiving the pre-transplant conditioning therapy, and/or administering the composition to reduce MHC class II expression on IEC in the transplant recipient at least two weeks prior to the transplant recipient receiving the pre-transplant conditioning therapy.

In some embodiments, the composition to modulate the transplant recipient's gut microbiome comprises an antibiotic.

In one embodiment, the antibiotic is a broad-spectrum antibiotic.

In one particular embodiment, the antibiotic is selected from vancomycin, polymixin B, amphotericin B, gentamycin, cefuroxime, and/or ceftriaxone.

In another embodiment, the composition to modulate the transplant recipient's gut microbiome comprises a probiotic or fecal transplant.

In one embodiment, the method comprises first administering an antibiotic to the transplant recipient, and then administering a probiotic and/or fecal transplant to the transplant recipient.

In another embodiment, the composition to reduce MHC class II expression on IEC in the transplant recipient comprises at least one of an inhibitor of an inflammatory cytokine, an innate defense regulator, Toll-like receptor inhibitor, an antimicrobial peptide, and/or a compound that depletes T-cells.

In one embodiment, the composition to reduce MHC class II expression on IEC in the transplant recipient comprises an inhibitor of an inflammatory cytokine.

In one embodiment, the inhibitor of an inflammatory cytokine reduces activity of an inflammatory cytokine selected from IL-12, IL-23 and/or IFN-γ.

In one particular embodiment, the inhibitor reduces activity of IL-12 and/or IL-23 in the transplant recipient.

In another embodiment, the inhibitor of the inflammatory cytokine is an antibody that binds to the inflammatory cytokine and/or reduces binding of the inflammatory cytokine to its receptor.

In one embodiment, the antibody binds to IL-12 and/or IL-23.

In one particular embodiment, the antibody binds to the p40 subunit of IL-12 and IL-23.

In one embodiment, the antibody is ustekinumab.

In another embodiment, the antibody is risankizumab.

In another embodiment, the inhibitor of the inflammatory cytokine is a compound that reduces cells within the GI tract that secrete one or more inflammatory cytokines.

In one embodiment, the one or more inflammatory cytokines are selected from IL-12, IL-23 and IFNγ.

In an embodiment, the cells within the GI tract are macrophages that secrete IL-12.

In another embodiment, the cells within the GI tract are innate type-1 lymphocytes (ILC1) that secrete IFNγ.

In yet another embodiment, the composition to reduce MHC class II expression on IEC in the transplant recipient comprises an innate defense regulator.

In one embodiment, the innate defense regulator is dusquetide.

In another embodiment, the composition to reduce MHC class II expression on IEC in the transplant recipient comprises an antimicrobial peptide.

In one embodiment, the compound that depletes T-cells is selected from an antithymocyte globulin and/or an anti-CD52 antibody.

In one particular embodiment, the anti-CD52 antibody is alemtuzumab.

In another embodiment, the compound that depletes T-cells is an antithymocyte globulin selected from thymoglobulin or ATGAM.

In one embodiment of the methods described herein, the method reduces the risk of the transplant recipient developing acute GVHD.

There is further provided a composition that modulates a transplant recipient's gut microbiome, and/or a composition that reduces MHC class II expression on IEC in a transplant recipient, for reducing the risk of the transplant recipient developing GVHD.

There is further provided use of a composition that modulates a transplant recipient's gut microbiome and/or a composition that reduces MHC class II expression on IEC in a transplant recipient in the manufacture of a medicament for reducing the risk of the transplant recipient developing GVHD.

There is further provided a pharmaceutical combination comprising

i) a composition that modulates a transplant recipient's gut microbiome; and
ii) a composition that reduces MHC class II expression on IEC in a transplant recipient.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

FIG. 1. Regulation of MHC class-II expression by intestinal epithelial cells in the GI tract. MHC class-II expression in the gut is enhanced by IL-12 and IFN-γ from macrophages and type I innate lymphoid cells respectively.

FIG. 2. IEC in the ileum express MHC class-II and is enhanced after total body irradiation (TBI). (A) Lethally irradiated Villin Cre^{pos}Rosa26-YFP mice (H-2^{b}) were transplanted with BM and T cells from BALB/c mice (H-2^{d}). On day 3, MHC class-II expression in the small and large intestine was analyzed together with nave mice. Representative FACS plots and enumeration are combined from 3 replicate experiments (n=9 per group). (B) Lethally irradiated B6D2F1 mice (H-2^{b/d}) were transplanted with BM and T cells from B6.WT mice (H-2^{b}). On day 4, MHC class-II expression in the small and large intestine was analyzed together with nave mice. Representative FACS plots and enumeration combined from 2 replicate experiments (n=4-5 per group). All statistical analysis by two-tailed Mann-Whitney test (means and SEM). *P<0.05. (C) Nave B6.MHCII-GFP mice, 24 h after TBI or d3 after BMT with BM and T cells from BALB/c mice as in (A).

FIG. 3. The intestinal microbiome drives MHC class-II expression on IEC. (A) Representative histograms of MHC class II MFI in ileum of specific pathogen free (SPF) and germ free (GF) mice before and 24 hrs after TBI. (B) Quantitation of MHC class II MFI as in A (n=6 per group from 3 experiments). (C) Representative confocal images of MHC class II expression as in A (from 3 experiments). (D) Representative histograms of MHC class II MFI in ileum of mice treated with antibiotic water (cefoxitin, vancomycin, gentamicin and metronidazole) control for 2 weeks and analyzed before and 24 hrs after TBI. (E) Quantitation of MHC class II MFI as in E (n=4-6 per group from 2 replicate experiments) (F, G) B6.WT and B6.IL-17RA−/− mice were housed individually or cohoused for 6 weeks and (F) Representative histograms MHC class-II expression on 7AAD^{neg}EpCAM⁺CD45^{neg} cells examined. (G) Quantitation of MHC class II MFI as in F (n=8-10 per group combined from 2 replicate experiments). ***P<0.001; ****P<0.0001. Two-way ANOVA test (means and SEM). (H) Cohoused or individually housed B6.WT mice were lethally irradiated and transplanted with CD4+ T cells from luciferase-expressing BALB/c mice. Day 7 Bioluminescence data (allogeneic CD4+ T cell expansion) shown are combined from 2 replicate experiments (n=9-10 per group). **P<0.01; ***P<0.001. Statistical analysis by two-tailed Mann-Whitney test (means, SEM).

FIG. 4. Pathogen-associated molecular patterns (PAMP) signaling drives MHC class-II expression and antigen presentation by IEC in the ileum. (A) Mixed Lymphocyte Reaction (MLR) of CFSE-labelled sort-purified Marilyn T cells, which possess male antigen (HY)-specific TCR expression, co-cultured with sort-purified IEC (7AAD^{neg}EpCAM⁺CD45^{neg}) cells from male or female small intestine 16-20 h post-TBI, analysed at day 7. Female IEC's co-culture are negative control (no HY antigen). Data are representative of 2 replicate experiments with n=3-5. (B) Epithelial cells from the small intestine (7AAD^{neg}Villin⁺CD45^{neg}) of nave Villin Cre^{pos}Rosa26-YFP mice, those 4d post-TBI or 3d post-BMT with/without T cells (GVHD/non-GVHD, respectively) were sort-purified and processed for RNAseq gene expression analysis (n=3-5 per group). First and second principal component projections revealed clustering of samples based on gene expression values with clear separation between naïve and all other sample types. (C) The epithelial fractions from naïve B6.WT and B6.MyD88^{-/-}TRIF^{-/-} mice were analyzed for MHC-II expression. Representative FACS plots and MFI are shown. Data are combined from 2 replicate experiments (n=7 per group). (D) Representative confocal microscopy of MHC-II expression on ileum before and 24 h after TBI with quantification normalized to naïve WT mice (n=3 per group). (E) Male B6.WT and B6.MyD88^{-/-}TRIF^{-/-} mice were transplanted with luciferase-expressing Marilyn HY-specific CD4+ T cells. WT female recipients were utilized as negative controls (n=4). Marilyn T cell quantification and Tbet expression in mesenteric LN and spleen. (n=5-8 per male group combined from 2 replicate experiments). *P<0.05; **P<0.01. Statistical analysis by two-tailed Mann-Whitney test (means, SEM).

FIG. 5. (A) IFNγ drives MHC-II expression on IEC. (A) Representative IFNγR and (B) MHC-II expression on IEC (7AAD^{neg}EpCAM⁺CD45^{neg}) from naïve and 24 h post-TBI, B6.WT and B6.IFNγ12^{-/-} mice. Representative histograms (left) and quantification (right) (n=6 per group, combined from 3 replicate experiments). (C) Representative MHC-II expression on IEC from naïve B6.WT, B6.Rag^{-/-}, B6.Rag^{-/-} cyc^{-/-} and B6.I-A^{b-/-} mice. Quantification shown right (n=4 per group from 3 experiments). (D) Representative IFNγ-eYFP expression (left) on the indicated populations from naïve mice and quantification within hematopoietic cells (right) are shown (n=3-6 per group from 2 replicate experiments).

FIG. 6. IEC induce MHC class-II-dependent GVHD. (A) Lethally irradiated B6 male Nestin, Villin or Tie2 Cre^{pos}I-A^{b-fl/fl} mice and relevant Cre^{neg}I-A^{b-fl/fl} mice were transplanted with BM from female I-A^{b} deficient mice (B6.I-A^{b-/-}). 3 months later, these chimeric mice were irradiated and transplanted with BM from female B6.I-A^{b-/-} mice with 25×10³ sorted Marilyn T cells. Survival by Kaplan-Meier analysis, combined from 2 replicate experiments (n=9-11 per group). P=0.0025 (Villin), 0.0066 (Nestin): Cre^{pos}I-A^{b-fl/fl} vs. Cre^{neg}I-A^{b-fl/fl} chimeric recipients. (B) BM chimeric recipients were transplanted as in (A) but with 0.2×10⁶ sorted luciferase-expressing Marilyn T cells. Bioluminescence data on day 7 are combined from 3-4 replicate experiments (n=12-21 per group). P=0.0067, Villin Cre^{pos}I-A^{b-fl/fl} vs. Villin Cre^{neg}I-A^{b-fl/fl} chimeric recipients. Two-tailed Mann-Whitney test (means and SEM shown).

FIG. 7. MHC-II-expressing IEC elicit alloantigen reactive T cell expansion and Th1 differentiation in the GI tract. Male (A-F) or female (G-H) Villin Cre-ER^{T2-neg}I-A^{b-fl/fl} and Cre-ER^{T2-pos}I-A^{b-fl/fl} or female B6.WT mice (A F) were treated with Tamoxifen 1 mg/day for 5 days, 2 weeks before transplant. (A) Lethally irradiated Tamoxifen-treated mice were transplanted with BM from female B6.WT mice with 0.5×10⁶ CD4 MACS-purified Marilyn T cells. Female recipients are negative controls. Survival by Kaplan-Meier analysis, combined from 2 replicate experiments (n=11-12 per T replete group). (B F) Recipients were transplanted as in (A) but with 0.2×10⁶ sorted luciferase-expressing Marilyn T cells. Serum TNF, bioluminescence data, enumeration of Marilyn T cells with MFI of T-bet expression and intestinal histology on day 7 are shown, combined from 3 replicate experiments (n=12 per T replete group). (G H) Lethally irradiated Tamoxifen-treated female mice were transplanted with BM and T_{reg}-depleted CD4⁺ T cells (5×10⁶ CD4 MACS-purified) from PC61-treated donor BALB/c mice. (G) Survival by Kaplan-Meier analysis, combined from 2 replicate experiments (n=11 per T replete group), (H) Intestinal histopathology scores on day 10 (n=6 per T replete group). All statistical analysis by two-tailed Mann-Whitney test (means and SEM) except for survival data (Kaplan-Meier estimates with log-rank comparisons).

FIG. 8. Neutralizing IL-12 prior to irradiation prevents the induction of MHC class-II expression by IEC and GVHD lethality. (A) Representative IL-12/23p40-eYFP on DC and macrophages (Mac) from nave B6.IL-12p40-YFP mice. B6.WT mice were gated as controls. (B) Quantification of IL-12/23p40YFP⁺ Mac and DC from nave mice or 6 h post-TBI (n=6-7 per group from 3 replicate experiments). (C) Fluorescence images of the ileum 24 h post-TBI. B6.WT mice were treated with IL-12p40 mAb or isotype control at 48 h and 24 h prior to TBI. Representative images are shown with quantification (n=5-6 per group from 2 replicate experiments). (D) Male B6.WT mice were treated with IL-12p40 mAb or isotype control at 48 h (day −3), 24 h (day −2) prior to TBI and the day of transplant (day 0) then transplanted with B6.WT bone marrow and Marilyn CD4 T cells (n=12, combined from 2 replicate experiments). Female recipients (n=4) were used as negative controls.

### DETAILED DESCRIPTION

### General Techniques and Definitions

Unless otherwise indicated, the recombinant protein, cell culture, and immunological techniques utilized in the present disclosure are standard procedures, known to those skilled in the art, such as those described in J. Perbal, A Practical Guide to Molecular Cloning, John Wiley and Sons (1984), J. Sambrook et al., Molecular Cloning: A Laboratory Manual, 3rd edn, Cold Spring Harbour Laboratory Press (2001), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including updates until present).

As used herein, the singular forms of “a”, “and” and “the” include plural forms of these words, unless the context clearly dictates otherwise.

The term “and/or”, e.g., “X and/or Y” shall be understood to mean either “X and Y” or “X or Y” and shall be taken to provide explicit support for both meanings or for either meaning.

As used here, the terms “treating”, “treat”, or “treatment” include administering a therapeutically effective amount of a pharmaceutical composition, for example, a pharmaceutical composition comprising an antibiotic, an anti-inflammatory, a probiotic and/or a fecal transplant to a patient sufficient to reduce or eliminate one sign or symptom of disease, and/or to reduce the risk of a patient developing one sign or symptom of a disease, for example graft-versus-host disease.

“Administering” as used herein is to be construed broadly and includes administering a composition or therapeutic agent as described herein to a subject or patient as well as providing the composition or therapeutic agent to a cell, such as, for example, by the provision of a prodrug to the subject or patient.

### Modulating Inflammation and MHC Class-II Expression in the Gut

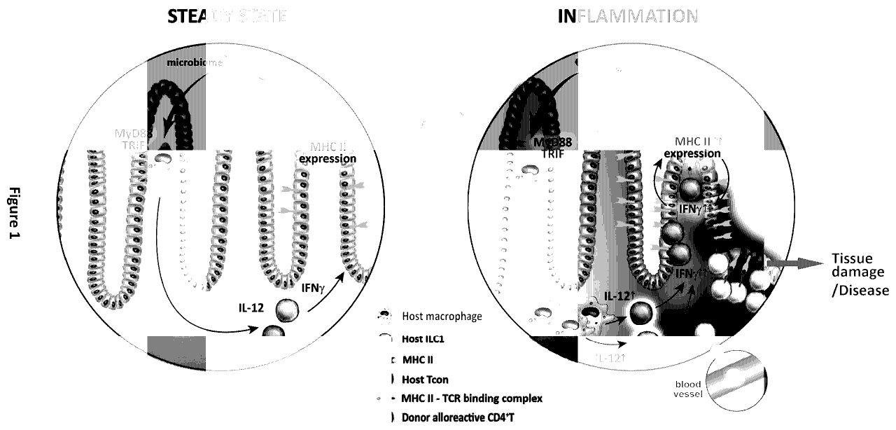

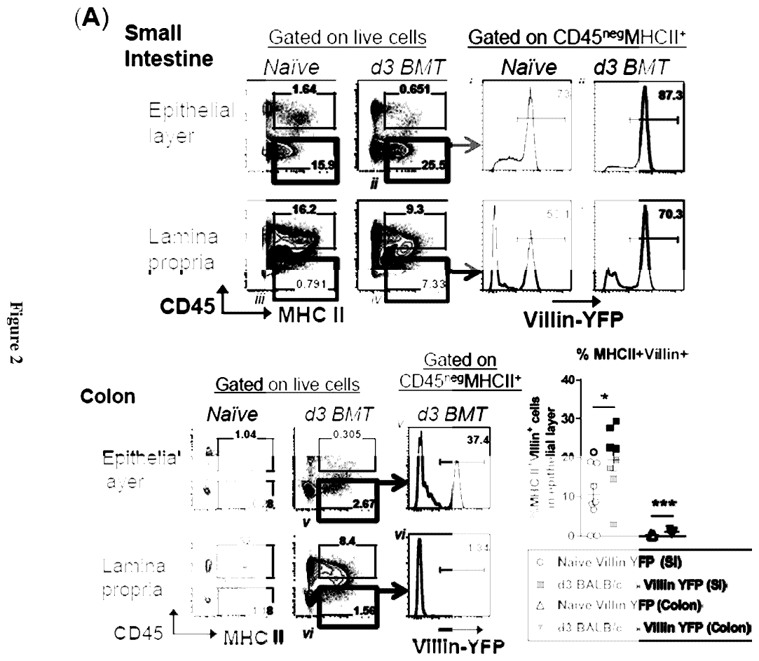

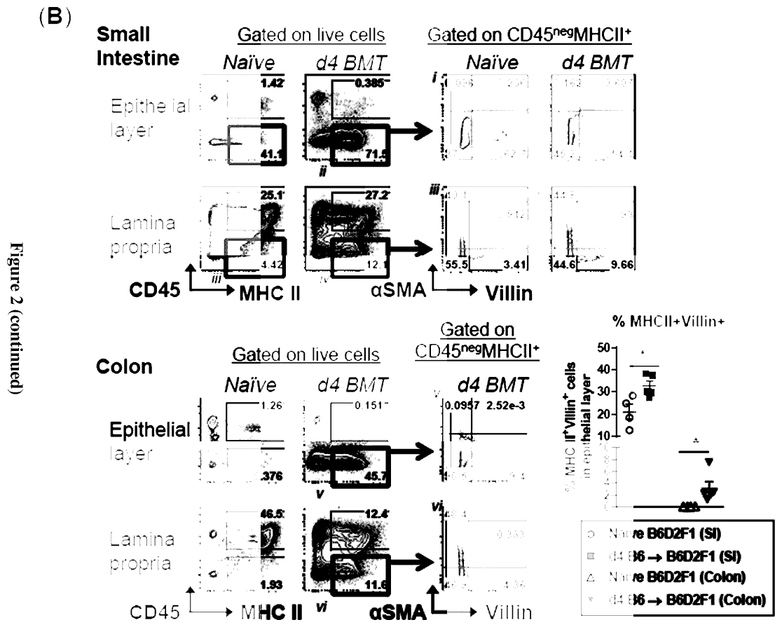

While not wishing to be bound by theory, the present disclosure relates to the finding that tight regulation of MHC class-II expression by intestinal epithelial cells in the GI tract is achieved by a balance of cytokine responses driven by macrophages and ILC1 immune cells under homeostatic conditions, and that pathogen-associated molecular patterns (PAMP) keep the epithelium poised to respond to challenges via antigen presentation. Further, during inflammation, MHC class-II expression is enhanced by IL-12 and IFN-γ from macrophages and conventional T-cells respectively (FIG. 1). The enhanced antigen presentation by intestinal epithelium drives immunopathology. These processes are absolutely microbiome-dependent as they were shown to be absent in germ-free mice.

In light of these findings, the present disclosure provides methods of reducing the risk of a transplant recipient developing graft-versus-host disease by administering a composition to a subject to modulate the subject's microbiome and/or administering a composition to reduce the activity of an inflammatory cytokine and/or to reduce expression of MHC II on intestinal epithelial cells in the subject. In order to reduce the risk of GVHD in a transplant recipient, the composition is administered to the subject before transplantation and preferably before conditioning therapy.

As understood in the art, conditioning therapy (also referred to as a conditioning regimen) is used to prepare a patient for stem cell transplantation. A conditioning therapy may include chemotherapy, monoclonal antibody therapy, and/or radiation to the entire body.

GVHD is a common complication following an allogeneic tissue transplant. It is commonly associated with stem cell or bone marrow transplant, but the term also applies to other forms of tissue graft. Immune cells (white blood cells) in the tissue (the graft) recognise the recipient (the host) as “foreign”. The acute or fulminant form of the disease (aGVHD) is normally observed within the first 100 days post-transplant and is a major challenge to transplants owing to the associated morbidity and mortality. Acute GVHD is characterised by selective damage to the liver, skin (rash), mucosa, and the gastrointestinal tract. Other GVHD target organs include the immune system (the hematopoietic system, e.g., the bone marrow and the thymus) itself, and the lungs in the form of idiopathic pneumonitis.

Acute GVHD is staged and graded (0-IV) by the number and extent of organ involvement. Patients with grade IV GVHD usually have a poor prognosis. If the GVHD is severe and requires intense immunosuppression involving steroids and additional agents to get under control, the patient may develop severe infections as a result of the immunosuppression and may die of the infection.

### Modulating the Microbiome

In some embodiments, the methods described herein are directed to modulating a patient's or transplant recipient's gut microbiome. As would be understood in the art, ‘modulating’ a patient's or transplant recipient's gut microbiome may include reducing or depleting the microflora in a subject. By reducing or depleting microflora in a subject, MHC-II antigen presentation and inflammatory processes in the gut may be inhibited, thus reducing the risk of a patient developing GVHD.

In other embodiments, it may be desirable to supplement and/or repopulate a subject's gut with beneficial bacteria, for example by administering a probiotic composition and/or fecal transplant to the subject. In some particular embodiments, it may be desirable to first deplete or reduce a subject's microbiome, for example to reduce or eliminate deleterious microflora by administering one or more antibiotic compositions to the subject, and then supplementing or repopulating the subject's gut microbiome with beneficial bacteria, such as may be provided in a probiotic composition or fecal transplant.

As used herein, the terms gut “microbiome”, “microbiota”, “microflora” and “flora” refer to a community of microbes that live in or on a subject's gut, both sustainably and transiently, including eukaryotes, archaea, bacteria, and viruses.

### Antibiotics

In some embodiments, the gut microbiome of a subject is modulated by administering antibiotics or other medications to which at least some of the microorganisms in the gut microbiome are sensitive. Such antibiotics and medications include antimicrobial compounds and antibiotics, including bacteriostatic and bacteriocidal compositions, as well as anti-viral medications and medications effective against protozoan and/or fungal pathogens.

Examples of antibiotics for modulating the gut microbiome, in particular for reducing the bacterial load in the gut, are well known in the art and include ansamycins, particularly rifamycins such as rifampin, rifabutin, rifapentine or rifamixin; fluoroquinolones, for example moxifloxacin; vancomycin, cephalosporin, linezolid, teicoplanin, abeaconazole, quinuclidine, streptogramins, particularly quinupristin and/or dalfopristin; and extracellular active antibiotics glycopeptides, particularly vancomycin or teicoplanin; fosfomycin; polypeptides, particularly bacitracin, daptomycin, or polymyxin B; and aminoglycosides, particularly arbekacin. Further examples include adapalene, tazarotene, erythromycin, clindamycin, azithromycin, minocycline, roxithromycin, isotretinoin, and benzoyl peroxide (BPO); amoxicillin, metronidazole, tinidazole, furazolidone, tetracycline and the like; or, the other conventional antibiotics are anti-fungal drugs including, but not limited to, clotrimazole, nystatin, fluconazole, Ketoconazole, itraconazole, miconazole, terbinafine, naftifine, amorolfine, amphotericin B, griseofulvin, ciclopirox olamine, caspofungin and the like.

### Probiotics

Probiotics are supplements or foods containing beneficial ‘probiotic’ microorganisms that naturally exist in the GI tracts of humans and animals. Oral administration of adequate amounts of probiotics may repopulate the gut with beneficial bacteria that may be missing in the subject.

Bacterial probiotics may include, but are not limited to bacteria such as Allobaculum, Lactobacillus species including Lactobacillus reuteri, Lactobacillus taiwanensis, Lactobacillus johnsonii, Lactobacillus animalis, Lactobacillus murinus, the genus Adlercreutzia, the phylum Actinobacteria, lactic acid bacterium, Lactobacillus bulgaricus, Streptococcus thermophiles, Bifidobacteria spp., Propionic acid bacteria, Bacteroides, Eubacterium, anaerobic Streptococcus, Lactobacillus delbrueckii subsp. Bulgaricus, Escherichia coli, other intestinal microorganisms.

In some examples, the probiotic compositions may comprise one or more of L. acidophilus, L. crispatus, L. gasseri, group L. delbrueckii, L. salivarius, L. casei, L. paracasei, L. plantarum, L. rhamnosus, L. reuteri, L. brevis, L. buchneri, L. fermentum, L. rhamnosus, B. adolescentis, B. angulation, B. bifidum, B. breve, B. catenulatum, B. infantis, B. lactis, B. longum, B. pseudocatenulatum, S. thermophiles, Pseudomonas fluorescens, P. protegens, P. brassicacearum, P. aeruginosa; Azospirillum brabrasilense, A. lipferum, A. halopraeferens, A. irakense; Acetobacter diazotrophicus; Herbaspirillum seropedicae; Bacillus subtilis, Pseudomonas stutzeri, fluorescens, P. putida, P. cepacian, P. vesicularis, P. paucimobilis; Bacillus cereus, B. thuringiensis, B. sphaericus; Shewanella oneidensis; Geobacter bemidjiensis, G. metallireducens, G. sulfurreducens, G. uraniireducens, G. lovleyi; Serratia marcescens, Desulfovibrio vulgaris, D. desulfuricans, Dechloromonas aromatic, Deinococcus radiodurans, Methylibium petroleiphilum, Alcanivorax borkumensis, Archaeglobus fulgidus, Haloferax sp., Halobacterium sp., and combinations thereof.

In some examples, the probiotic composition may be used in conjunction with a prebiotic composition. As would be understood in the art, prebiotic substances may help to stimulate the growth of the probiotic organisms. Prebiotic compositions may contain, for example, ingredients such as a water-soluble carbohydrate, inulin, oligosaccharides, oligofructose, fructo-oligosaccharide, galacto-oligosaccharide, glucose, starch, maltose, maltodextrins, polydextrose, amylose, sucrose, fructose, lactose, isomaltulose, polyols, glycerol, carbonate, thiamine, choline, histidine, trehalos, nitrogen, sodium nitrate, ammonium nitrate, phosphorus, phosphate salts, hydroxyapatite, potassium, potash, sulfur, homopolysaccharide, heteropolysaccharide, cellulose, chitin, vitamins, and combination thereof.

### Fecal Microbiota Transplantation

Transplantation or administration of human colonic microbiota into the bowel of a patient is called Fecal Microbiota Transplantation (FMT), also commonly known as fecal bacteriotherapy. FMT is believed to repopulate the gut with a diverse array of microbes that control key pathogens by creating an ecological environment inimical to their proliferation and survival. It represents a therapeutic protocol that allows a fast reconstitution of a normal compositional and functional gut microbial community.

As used herein, a “fecal microbiome” or “fecal microbiome preparation” refers to a community of microbes present in a subject's feces. A non-selected fecal microbiome refers to a community or mixture of fecal microbes derived and processed from a donor's fecal sample without selection for any particular group or type of microbes and substantially resembling microbial constituents and population structure found in such fecal sample.

Typically, fecal transplant material is derived from healthy donors who have no risk factors for transmissible diseases and have not been exposed to agents, such as, for example, antibiotics, that could alter the composition of their gut microbiota.

Details pertaining to the harvesting and processing of FMT material are known in the art. Briefly, many protocols call for use of fresh feces, which requires collection and processing on the same day scheduled for the FMT. Other protocols have been developed that use highly filtered human microbiota mixed with a cryoprotectant, which can be frozen for storage at −80° C. until required for use (Hamilton et al., 2012). This approach benefits from convenience with regard to scheduling, and generates a processed fecal material (fecal filtrate) having reduced volume and fecal aroma. Equivalent clinical efficacy has been noted when either purified processed fecal material or fresh, partly filtered feces were used. FMT material may be administered via naso-duodenal, transcolonoscopic, or enema based routes.

### Modulating Activity of Inflammatory Cytokines and Inhibiting MHC II Expression

In some embodiments of the methods described herein, the method comprises administering to a subject a composition comprising an inhibitor of an inflammatory cytokine in order to inhibit MHC-II antigen presentation and/or inflammatory processes in the gut.

### Inhibitors of Inflammatory Cytokines

In some examples of the methods described herein, reducing the risk of a transplant recipient developing GVHD comprises administering a composition to the transplant recipient or subject to reduce activity of an inflammatory cytokine in the transplant recipient or subject. For example, the method may comprise administering a composition to reduce activity of an inflammatory cytokine selected from IL-12, IL-23, IFN-γ, IL-1 and/or IL-6.

The compositions that reduce activity of an inflammatory cytokine may contain a compound that binds directly to the cytokine, for example such as a small molecule inhibitor or antibody that binds directly to the cytokine (e.g. an anti-IL-12 antibody that reduces activity of IL-12). Alternatively, the composition may contain a compound that binds to a molecule other than the cytokine and which exerts its effect of reducing the activity of the cytokine indirectly. For example, the composition may contain a compound that acts on another molecule in a signalling pathway of the cytokine and which indirectly reduces activity of the cytokine, for example by blocking signal transduction. Thus, in one example, the compound is a small molecule inhibitor or antibody that binds to an inflammatory cytokine receptor such as the IL-12 receptor or the IFN-γ receptor.

Interleukin 12 (IL-12) is in interleukin that is naturally produced by dendritic cells, macrophages, neutrophils, and human B-lymphoblastoid cells in response to antigenic stimulation. IL-12 is composed of a bundle of four alpha helices. It is a heterodimeric cytokine encoded by two separate genes, IL-12A (p35) and IL-12B (p40). The active heterodimer (referred to as ‘p70’), and a homodimer of p40 are formed following protein synthesis. IL-12 is involved in the differentiation of naive T cells into Th1 cells. It is known as a T cell-stimulating factor, which can stimulate the growth and function of T cells. It stimulates the production of interferon-gamma (IFN-γ) and tumor necrosis factor-alpha (TNF-α) from T cells and natural killer (NK) cells, and reduces IL-4 mediated suppression of IFN-γ.

Antibody inhibitors of IL-12 are known in the art and include Briakinumab (ABT-874), a human monoclonal antibody developed by Abbott Laboratories, and Ustekinumab, which is a human monoclonal antibody sold under the brand name Stelara and which is used to treat psoriasis. Ustekinumab binds to the p40 subunit of IL-12 and prevents IL-12 from binding to its receptor.

In some embodiments, the composition that reduces activity of an inflammatory cytokine may comprises an inhibitor of IL-12/23. As understood in the art, IL-12 and IL-23 share the p40 subunit. Thus, inhibitors active against the p40 subunit may inhibit the activity of both IL-12 and IL-23, for example such as antibodies that bind the p40 subunit. In other embodiments, the composition that reduces activity of an inflammatory cytokine may comprise an inhibitor that inhibits the activity of IL-23 and not IL-12.

IFN-γ is a dimerized soluble cytokine that is critical for innate and adaptive immunity against viral, some bacterial and protozoal infections. IFN-γ is an important activator of macrophages and inducer of Class II major histocompatibility complex (MHC) molecule expression. Aberrant IFN-γ expression is associated with a number of auto-inflammatory and autoimmune diseases. The importance of IFN-γ in the immune system stems in part from its ability to inhibit viral replication directly, and most importantly from its immunostimulatory and immunomodulatory effects. IFN-γ is produced predominantly by natural killer (NK) and natural killer T (NKT) cells as part of the innate immune response, and by CD4 Th1 and CD8 cytotoxic T lymphocyte (CTL) effector T cells once antigen-specific immunity develops. IFN-γ is also produced by non-cytotoxic innate lymphoid cells (ILC).

IFN-γ inhibitors include small molecules and antibodies that bind IFN-γ, for example such as the antibody AMG 811 described in US patent publication No. 20120269819. Another example of an anti-IFN-γ antibody is NI-0501, a monoclonal antibody that neutralises IFN-γ activity.

### Innate Defense Regulators and Host Defense Peptides

In some embodiments, compounds that inhibit inflammatory cytokines and/or which reduce MHC II expression on intestinal epithelial cells may comprise an innate defense regulator, host defence peptide and/or an antimicrobial peptide. Innate defense regulators (IDR) are a family of short, synthetic, peptide and peptide-like analogs with a dual-mode of efficacy, modulating the innate immune response to both damage and pathogen-associated signals, enhancing resolution of infection and tissue damage while suppressing harmful inflammation. IDR peptides are synthetic variants of naturally occurring host defense peptides (HDP). Naturally-occurring HDPs are cationic amphipathic molecules with immunomodulatory and microbicidal properties. Many HDPs have been characterized of which the defensins and cathelicidins have been of greatest focus. There are more than 1500 HDPs (http://aps.unmc.edu/AP/main.php) currently identified which have provided templates for designing short synthetic peptides, using internal fragments or amino acid substitutions, for designing IDR peptides; and using known methods, IDR peptides have been developed and optimized to exhibit improved immune-modulatory properties and reduced cytotoxicities compared to the parent HDP. One example of an innate defense regulator is dusquetide (SGX942) (North et al., 2016), which is understood to modulate the innate immune response to both PAMPs and DAMPs.

Antimicrobial peptides (AMPs), also referred to as host defense peptides (HDPs), are part of the innate immune response. Antimicrobial peptides have been demonstrated to kill Gram negative and Gram positive bacteria, enveloped viruses, and fungi. Antimicrobial peptides are a unique and diverse group of molecules, which are divided into subgroups on the basis of their amino acid composition and structure. Antimicrobial peptides are generally between 12 and 50 amino acids. These peptides include two or more positively charged residues provided by arginine, lysine or, in acidic environments, histidine, and a large proportion (generally >50%) of hydrophobic residues. In addition to killing bacteria directly they have been demonstrated to have a number of immunomodulatory functions that may be involved in the clearance of infection, including the ability to alter host gene expression, act as chemokines and/or induce chemokine production, inhibiting lipopolysaccharide induced pro-inflammatory cytokine production, promoting wound healing, and modulating the responses of dendritic cells and cells of the adaptive immune response. Antimicrobial peptides have been used as therapeutic agents, including bacitracin, boceprevir, dalbavancin, daptomycin, oritavancin, teicoplanin, telavancin, vancomycin and guavanin 2.

The antimicrobial peptide may be any antimicrobial peptide known to the person skilled in the art. In one example, the antimicrobial peptide is selected from the group consisting of a cationic or polycationic peptide, an amphipatic peptide, a sushi peptide, a defensin and a hydrophobic peptide. In some examples, the antimicrobial peptide is selected from the group consisting of an acidocin, actagardine, agrocin, alveicin, aureocin, aureocin A53, aureocin A70, carnocin, carnocyclin circularin A, colicin, Curvaticin, divercin, duramycin, Enterocin, enterolysin, epidermin/gallidermin, erwiniocin, gassericin A, glycinecin, halocin, haloduracin, lactocin S, lactococin, lacticin, leucoccin, lysostaphin macedocin, mersacidin, mesentericin, microbisporicin, microcin S, mutacin, nisin, paenibacillin, planosporicin, pediocin, pentocin, plantaricin, pyocin, reutericin 6, sakacin, salivaricin, subtilin, sulfolobicin, thuricin 17, trifolitoxin, variacin, vibriocin, warnericin and a warnerin.

### Toll-Like Receptor Inhibitors

Toll-like receptors (TLRs) are a family of pattern recognition receptors (PRRs) that can recognize and respond to a unique repertoire of distinct molecules referred to as pathogen-associated molecular patterns (PAMPs) and danger-associated molecular patterns. In general, TLR inhibition can be achieved by two major strategies: (1) blocking the binding of TLR ligands to the receptor; (2) interfering the intracellular signaling pathways to stop the signal transduction. Various therapeutic agents for inhibiting TLR signaling have been developed to control excessive inflammation; they can be classified as small molecule inhibitors, antibodies, oligonucleotides, lipid-A analogs, microRNAs, and new emerging nano-inhibitors.

Various therapeutic agents for inhibiting TLR signaling have been developed to control excessive inflammation; they can be classified as small molecule inhibitors, antibodies, oligonucleotides, lipid-A analogs, microRNAs, and new emerging nano-inhibitors. Antibodies can be designed to neutralize soluble effectors, block the binding of receptors to their ligands to stop signal transduction, or induce targeted cytotoxicity. In terms of inhibiting TLR signaling, antibodies are designed (as antagonists) to block the binding of ligands to the specific TLRs.

TLR antagnonists/inhibitors are known to include, for example, TAK-242, Candersartan, valsartan, fluvastatin, simvastatin, atorvastatin, ST2825, CQ HCQ, Cpg-52364, SM934, OPN-305, T2.5, NI-0101, 1A6, IRS-954, DV-1179, IMO-3100, IMO-8400, IMO-9200, IHN-ODN2088, IHN-ODN-24888, Eritoran, miR-146a, and miR-21.

Reducing Cells within the GI Tract that Secrete Inflammatory Cytokines

In some embodiments, a composition to reduce or eliminate cells within the GI tract that express inflammatory cytokines may be administered to the transplant recipient. The cells in the GI tract may be those that express inflammatory cytokines such as IL-12, IL-23 or IFNγ. Compounds that may reduce or eliminate cells within the GI tract that express inflammatory cytokines include antibodies or small molecules targeting macrophage surface proteins, their phagocytic function or cytokines critical for their survival (for example, clodronate loaded liposomes, or monoclonal antibodies to CSF-1R). Alternatively, the compound may be an antibody or small molecule targeting ILC1 surface proteins or cytokines critical for their survival (for example, antibodies to CD200r, CD52 or polyclonal anti-T cell mAb (ATG)).

### Compositions

The present disclosure describes compositions suitable for use in reducing the risk of a transplant recipient developing graft versus host disease. Compositions according to the present disclosure may comprise one or more gut microbiome modulators and/or compositions that reduce MHC class II expression on intestinal epithelial cells (IEC) in a transplant recipient, together with one or more pharmaceutically acceptable carriers, diluents and/or excipients. If desired, other active ingredients, adjuvants and/or immunopotentiators may be included in the compositions.

The compositions can be formulated for administration by a variety of routes. For example, the compositions can be formulated for oral, topical, rectal, nasal or parenteral administration or for administration by inhalation or spray. The term parenteral as used herein includes subcutaneous injections, intravenous, intramuscular, intrathecal, intrasternal injection or infusion techniques.

The compositions preferably comprise an effective amount of the one or more gut microbiome modulators and/or compositions that reduce MHC class II expression on intestinal epithelial cells (IEC) in a transplant recipient. The term “effective amount” as used herein refers to an amount of the composition required to reduce, inhibit or downregulate MHC class II expression on IEC. The effective amount can be estimated initially, for example, either in cell culture assays or in animal models, usually in rodents, rabbits, dogs, pigs or primates. The animal model may also be used to determine the appropriate concentration range and route of administration. Such information can then be used to determine useful doses and routes for administration in the animal to be treated, including humans.

Compositions for oral use can be formulated, for example, as tablets, troches, lozenges, aqueous or oily suspensions, dispersible powders or granules, emulsion hard or soft capsules, or syrups or elixirs. Such compositions can be prepared according to standard methods known to the art for the manufacture of pharmaceutical compositions and may contain one or more agents selected from the group of sweetening agents, flavoring agents, coloring agents and preserving agents in order to provide pharmaceutically elegant and palatable preparations. Tablets may contain an active agent in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

The compositions can be formulated as a sterile injectable aqueous or oleaginous suspension according to methods known in the art and using suitable one or more dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer's solution, lactated Ringer's solution and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, Pa. (2000).

### Kits

The present disclosure additionally provides for kits for reducing the risk of a transplant recipient developing GVHD, the kits comprising one or more gut microbiome modulators and/or one or more compositions that reduce MHC class II expression on intestinal epithelial cells (IEC) in a transplant recipient. Individual components of the kit would typically be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the medicament.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, nasal spray device, syringe, pipette, forceps, measured spoon, eye dropper or similar medically approved delivery vehicle.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

### Example 1. Materials and Methods

Mice. C57BL/6J (B6.WT, H-2^{b}, CD45.2⁺), BALB/c (H-2^{d}, CD45.2⁺) and B6D2F1 (H-2^{b/d}, CD45.2⁺) were purchased from the Animal Resources Centre, Perth, AU. Transgenic and knockout mice on a B6 background originated as follows: H2-Ab1^{-/-}, referred to as B6.1-A^{b-/-}, Australian National University, Canberra, AU; Villin Cre-ER^{T2}, Dr R Blumberg, Harvard Medical School, Boston, Mass., USA (Adolph et al., 2013; el Marjou et al., 2004); Rosa26-YFP, Dr B. Stockinger, MRC National Institute for Medical Research, Mill Hill, London, UK (Hirota et al., 2011); β-actin-luciferase, Dr Robert Negrin, Stanford, Calif., USA (Beilhack et al., 2005); Marilyn (Rag2^{-/-} background), Dr P Matzinger, NIH, Bethesda, Md., USA (Lantz et al., 2000); MHC class-II.EGFP, Dr Barbara Fazekas de St Groth, Garvan Institute, Sydney, AU; 2011); B6.MyD88^{-/-}TRIF^{-/-}, Dr S Akira, Osaka University, Osaka, Japan (Yamamoto et al., 2003); I-A^{b-fl/fl} (Stock 013181, B6.129X1-H2-Ab1^{tm1Koni}/J) (Hashimoto et al., 2002), Villin-Cre (Stock 004586, B6.SJL-Tg(Vil-cre)997Gum/J), Nestin-Cre (Stock 003771, B6.Cg-Tg(Nes-cre)1Kln/J), Tie2-Cre (Stock 008863, B6.Cg-Tg(Tek-cre)1Ywa/J), B6.IL-12p40-YFP (Stock 006412, B6.129-I112b^{tm1Lky}/J), B6.IFN-γ-YFP (Stock 17581, B6.129S4-Ifng^{tm3.1Lky}/J) and B6.IFNγ12^{-/-} (Stock 003288, B6.129S7-Ifngr1^{tm1Agt}/J), the Jackson Laboratory, Bar Harbor, Mass., USA. Rag2^{-/-} background Marilyn mice were backcrossed onto a B6 β-actin-luciferase background (Marilyn^{luc+}, CD90.1⁺CD45.2⁺). Each Cre strain and I-A^{b-fl/fl} or Rosa26-YFP strains were intercrossed to generate Villin-Cre.I-A^{b-fl/fl}, Villin-Cre-ER^{T2}I-A^{b-fl/fl}, Nestin-Cre I-A^{b-fl/fl}, Tie2-Cre I-A^{b-fl/fl}, Villin-Cre Rosa26-YFP, Nestin Cre Rosa26-YFP and Tie2 Cre Rosa26-YFP mice. Mice were bred at the QIMR Berghofer animal facilities. Mice were housed in sterilized micro-isolator cages and received acidified autoclaved water (pH 2.5) after transplantation. Experiments were performed with sex and age-matched animals using littermates where possible.

Stem cell transplantation. B6 or B6D2F1 mice were transplanted as described previously (Koyama et al., 2012) with 1000 cGy or 1100 cGy total body irradiation (TBI, 137Cs source at 84 cGy/min) on day −1, respectively. On day 0, B6 mice were transplanted with 5×10⁶ bone marrow (BM) cells and (0.025-0.5×10⁶) luciferase-expressing Marilyn cells. BM cells were depleted of T cells by antibody and complement as previously described (T cell depletion=TCD). Marilyn cells (Rag2^{-/-} background) were purified to greater than 98% by sorting of Vβ6⁺ CD8^{neg} cells using a MoFlo (Beckman Coulter) or 70% by CD4 MACS system (Miltenyi). To generate MHC II deficient BM chimeric mice, the relevant Cre-^{neg}I-A^{b-fl/fl} and Cre-^{pos}I-A^{b-fl/fl} mice treated with anti-CD4 (GK1.5) and CD8 (53-5.8) mAbs were injected with 10×10⁶ female B6.1-A^{b-/-} BM. The Ab were administrated i.p. from day −2 (GK1.5; 500 μg/animal, 53-5.8; 150 μg/animal) followed by weekly injection (GK1.5; 250 μg/animal, 53-5.8; 150 μg/animal) until week 6 to prevent any persistence of recipient T cells in a class II negative environment. Five weeks after Ab discontinuation, the BM chimeric mice were used in secondary transplantation. For BMT utilizing WT polyclonal CD4⁺ T cells, lethally irradiated female mice were transplanted with 5×10⁶ BM and 5×10⁶ CD4+ T cells (MACS purified) from Treg-depleted BALB/c donor mice (PC61; 500 μg/animal, day −3 and −1, i.p.). Tamoxifen 1 mg/day was administered for 5 days, 2 weeks before transplant where indicated. IL-12p40 (C17.8) or control rat IgG2a (MAC4) was administered intraperitoneally at 500 μg per dose, days −2 and −1 prior to TBI. In the BMT using B6D2F1 recipients, lethally irradiated female B6D2F1 mice were transplanted with 5×10⁶ BM and 2×10⁶ T cells using magnetic bead depletion as previously described (MacDonald et al., 2010). The severity of systemic GVHD was assessed by scoring as previously described (maximum index=10) (Cooke et al., 1996). For survival experiments, transplanted mice were monitored daily and those with GVHD clinical scores ≥6 were sacrificed and the date of death registered as the next day, in accordance with institutional guidelines.

Cell isolation from small intestine and colon. Longitudinally sectioned pieces of the small intestine or the colon were processed using a gentleMACS Dissociator and mouse lamina propria dissociation kit (both Miltenyi Biotec), according to the manufacturer's protocol. Dithiothreitol was excluded from the entire process.

FACS analysis. For surface staining, cell suspensions were incubated with anti-CD16/CD32 (2.4G2) followed by staining with antibodies against CD45.2, CD31, Ter119, MHC class II (I-A/I-E), CD69, EpCAM (CD326), CD19, CD3, TCRβ, TCRγδ, CD4, CD8, NKp46, Ly6G, CD11c, CD64, CD11b and T-bet (all BioLegend); CD90.1 and CD200r (eBioscience); V136, CD19, DX5 (CD49b), NK1.1, Ly6C and IFNγR1 (CD119) (BD Biosciences). 7AAD (Sigma) was added before FACS acquisition. For intracytoplasmic staining (villin and vimentin (Abcam, Cambridge, UK) and αSMA (eBioscience)), single-cell suspensions with or without YFP were incubated with fixable live/dead cell dye (ThermoFisher), FcγR blocked with 2.4G2 and stained for surface markers. They were then fixed and permeabilized using the BD Fix/Perm kit (BD) according to the manufacturer's protocol and stained with Abs against villin, vimentin or αSMA for 30 min at room temperature, washed and data acquired. T-bet expression was determined using the ICC Foxp3 staining buffer set (eBioscience) according to the manufacturer's protocol. Samples were acquired with a BD LSRFortessa (BD) and analysis was performed with FlowJo v9 (Tree Star) software.

Histologic analysis. H&E stained sections of formalin-fixed tissue were coded and examined in a blinded fashion by A.D.C. using a semi-quantitative scoring system, as previously described (Koyama et al., 2012). Images of GVHD target tissues were acquired using an Olympus BX51 microscope (Olympus), an Evolution MP 5.0 Camera, and Qcapture software (Qimaging).

Immunofluorescence microscopy. Tissues were fixed with 4% paraformaldehyde then placed in 30% sucrose prior to being frozen in Tissue-Tek OCT compound (Sakura Finetek). Sections (7 μm thickness) were treated with Background Sniper (Biocare Medical) and 2% BSA for 30 min and then stained with IA/IE PE (isotype Rat IgG2b PE) and CD45-Alexaflour 647 (isotype Rat IgG2b AF647) (all mAbs; BioLegend) for 120 minutes at room temperature in the dark. After washing, sections were counterstained with DAPI for 5 min and cover-slipped with Vector Vectashield Hard Set mounting media. Samples for quantitation of mucus thickness were prepared as described (Johansson et al., 2008). In brief, samples were fixed in Methacarn (60% methanol, 30% chloroform, 10% glacial acetic acid, Sigma) for 6 h, washed in methanol followed by ethanol and xylol, and embedded in paraffin. Slides were deparaffinized and washed in ethanol. In situ hybridization was done at 46° C. for 16 h in a hybridization buffer (0.9 M NaCl, 20 mM Tris/HCl, 0.01% sodium dodecyl sulfate (SDS), 20% formamide) containing a eubacterial probe Eub338 (Cy3-GCTGCCTCCCGTAGGAGT (SEQ ID NO:1)) and non-Eub (6-FAM-ACTCCTACGGGAGGCAGC (SEQ ID NO:2)) (each 10 ng/μl). Slides were washed in washing buffer (0.225 M NaCl, 20 mM Tris/HCl, 0.01% SDS, 5 mM EDTA) for 15 min. Slides were then washed in PBS and blocked in PBS containing 4% FCS. Polyclonal Muc2 antibody was applied for 4-16 h at 4° C. followed by incubation with secondary anti-rabbit Alexa Fluor 647 antibody for 1-2 h at 4° C. and DAPI for counterstaining. Images were taken with ×40 oil-immersion lens or ×10 non-oil lens using a Zeiss 780-NLO Point Scanning Confocal Microscope with Zen software (Zen software).

Mixed lymphocyte reaction. Carboxy fluorescein diacetate succinimidyl ester (CFSE) labelling was performed as described (Koyama et al., 2015). In round-bottom 96-well plates, sort-purified CFSE-labeled Marilyn^{luc+} cells (CD90.1⁺) were stimulated with sort-purified intestinal epithelial cells (IEC) (CD326⁺CD45.2^{neg}) from the small intestine of female or male B6.WT mice which had undergone TBI 20 h before, in the presence of rhIL-2 (100 U/ml)(Zhang et al., 2017). Seven days later, Marilyn^{luc+} T cells (CD90.1⁺CD4⁺CD45.2⁺) were assessed for CFSE dilution and expression of CD69 by FACS.

Cytokine analysis. Serum TNF levels were determined using the BD Cytometric Bead Array system (BD Biosciences Pharmingen) according to the manufacturer's protocol.

Bioluminescence imaging (BLI). T cell expansion was determined by analysis of luciferase signal intensity (Xenogen IVIS 100; Caliper Life Sciences). Light emission is presented as photons per second per square centimetre per steer radiant (ph/s/cm2/sr). Total flux of mouse or organ is presented as photons per second (ph/s). Mice were subcutaneously injected with 500 μg d-Luciferin (PerkinElmer) and imaged 5 min later.

RNA sequencing. Total RNA was extracted from sort-purified IEC (CD45^{neg}Villin-YFP⁺ 7AAD^{neg}) from VillinCre⁺Rosa26YFP mice using the RNeasy mini kit (QIAGEN). For library preparation and sequencing, TruSeq Stranded Total RNA Ribo-Zero GOLD and NextSeq 75 cycle High output run (Illumina) were utilized, respectively. We compared IEC from nave mice, those from mice 4 days after TBI but not transplanted, those from transplanted (BALB/c→VillinCre⁺Rosa26YFP) non-GVHD mice (i.e. transplanted with TCD grafts) and those from GVHD mice (transplanted with T cell replete grafts). Sequence reads were trimmed for adapter sequences using Cutadapt and aligned to the mm10 assembly using STAR aligner. The read counts per gene were estimated using RSEM and were utilized to determine differential gene expression between groups using Bioconductor package ‘edgeR’. The default TMM normalization method of edgeR was used to normalise read counts between samples. Differentially expressed genes were considered significant if the Benjamini-Hochberg corrected p-value was less than 0.01. Gene set enrichment analysis (GSEA 3.0, Broad Institute) was used to determine genesets and pathways that are significantly enriched in up and down differentially expressed genes (fold change >2, fdr<0.01) for each group of comparison against the GSEA molecular signature database. The genesets that were significant with an FDR<0.05 and are common in all comparisons from the above GSEA analysis were utilised for single-sample GSEA (ssGSEA) analysis. ssGSEA analysis reveals the pathways that are differentially regulated between the samples. The sample projection values for each pathways from ssGSEA analysis were used to construct a heat map demonstrating changes in pathway enrichments for each sample.

Statistical analysis. Data were analysed using GraphPad Prism (ver. 7.02). Survival curves were plotted using Kaplan-Meier estimates and compared by log-rank test. If the equality of variance tests indicated the group variances were not significantly different (p>0.01), ANOVA (two-way) was used. The Mann Whitney-U test was used for the statistical analysis of remaining data when comparing two groups. Data are presented as mean±standard error of the mean (SEM).

### Example 2. Results

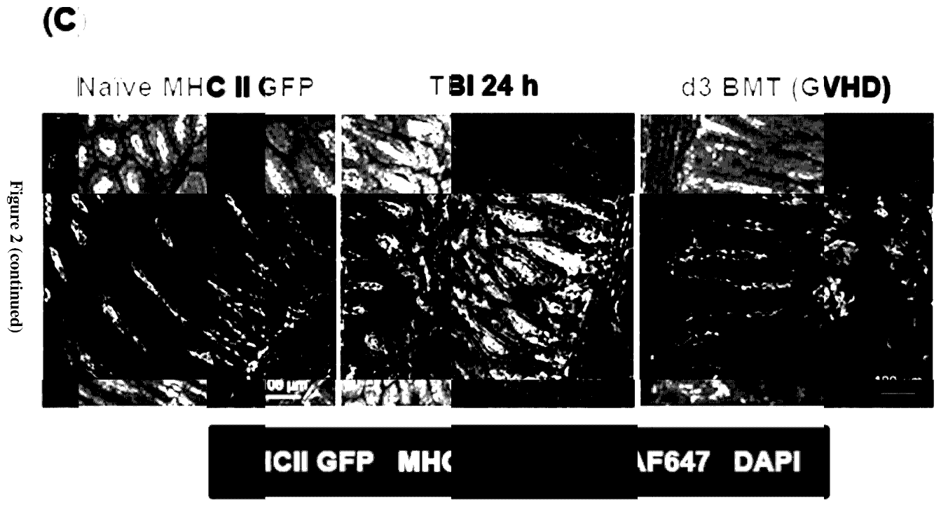

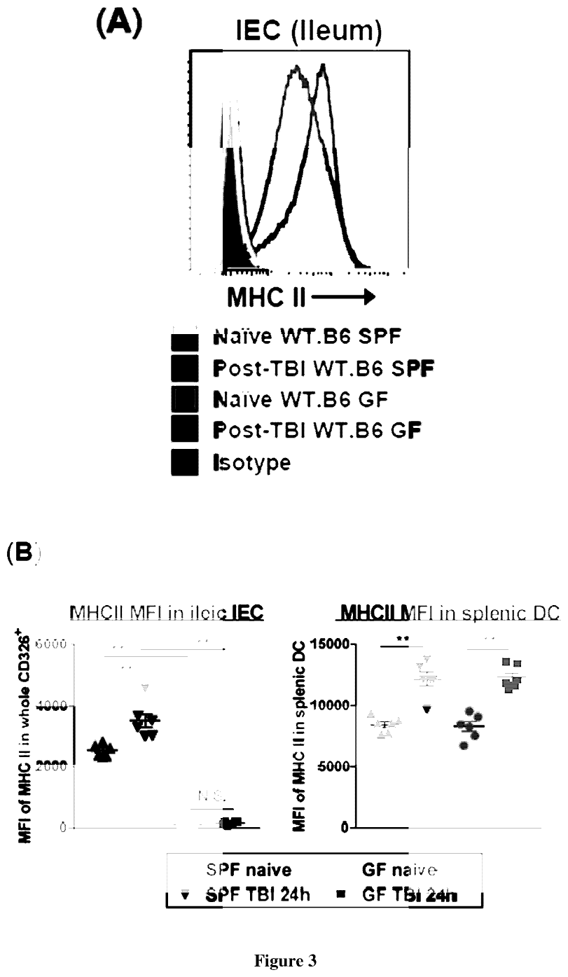

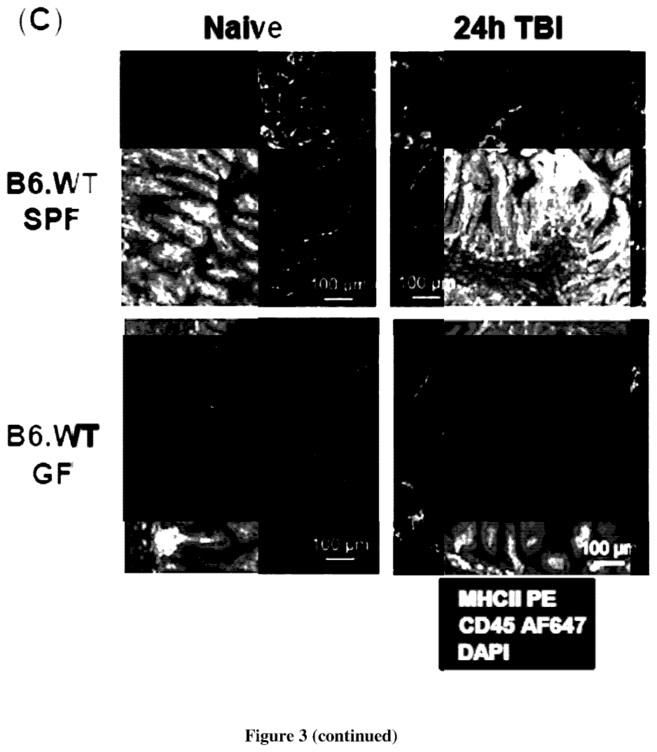

IEC in the ileum express MHC class-II and present alloantigen to CD4⁺ cells. The factors initiating immune pathology in the GI tract are unclear but atypical antigen presentation is likely involved. The nature and relative importance of antigen presenting cells (APC) that operate in the GI tract is unknown, as is their relevance under physiological steady-state and inflammatory conditions. An analysis of MHC class-II expression in various components and anatomical sites of the GI tract was performed and showed that, surprisingly, MHC II was highly and preferentially expressed in the ileum at steady state (FIG. 2A B). Early after BMT, expression of MHC class-II increased in the small intestine and, to a lesser extent, the colon (FIG. 2A B). Confocal microscopy of the GI tract in recipients where the MHC class-II promoter drives GFP confirmed a dramatic up-regulation of MHC class-II expression after conditioning with Total Body Irradiation (TBI) (FIG. 2C). The distribution of MHC class-II GFP⁺ cells was found to overlap with that of MHC class-II Ab-stained cells.

The differential distribution of MHC class-II in various sections of the GI tract suggested that there may be tissue site specific differences that dictate IEC responses. It has been previously demonstrated by 16s sequencing of the microbiome in GI tract tissue that bacterial translocation occurs preferentially at the ileum. Comparing the inner mucus of the colon before and early after BMT, it was found that the inner mucus layer which shields IEC from bacteria remained intact in the first days following TBI (not shown). In contrast to the impenetrable inner mucus layer of the colon, the mucus layer in the small intestine is less dense and more permeable to small sized molecules. It was found that bacteria were present just above the villi in direct contact with the IEC of the ileum, where an inner mucus layer was absent (not shown). These findings support the concept that the terminal ileum is a preferential site of mucosal-microbiome interaction, afforded by the presence of a limited mucus layer relative to that of the colon.

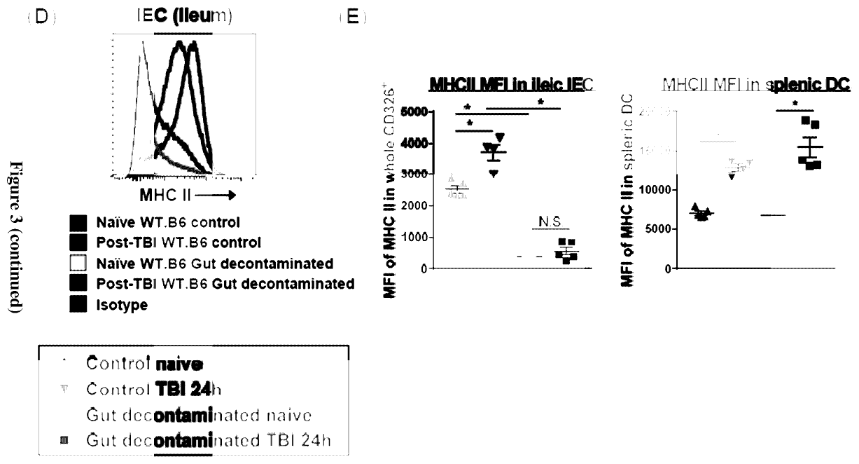

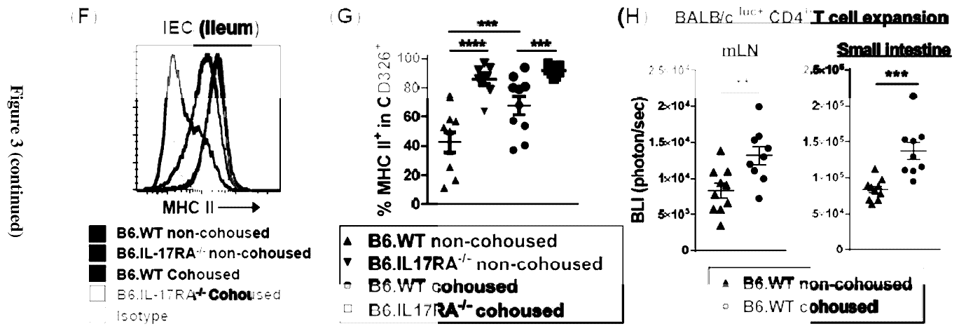

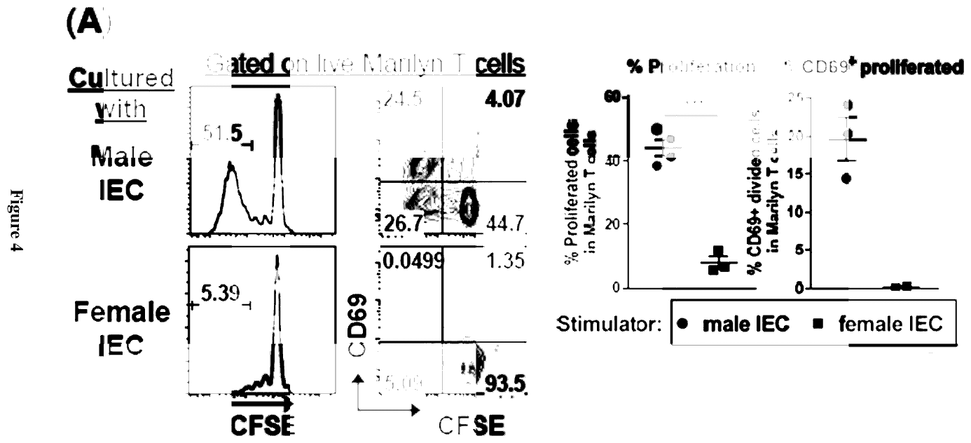

The microbiome drives MHC class-II expression by IEC in the small intestine. The role of the microbiome and relevant DAMP signals in driving MHC class II expression by IEC was investigated. MHC expression in germ free mice was analysed and it was noted that MHC class II was completely absent in the IEC of these animals, even after TBI (FIG. 3A C). In contrast, MHC class II expression by hematopoietic APC was intact and was upregulated after TBI normally in germ free mice (FIG. 3B). To confirm these results, the GI tract was decontaminated with broad spectrum oral antibiotics (vancomycin, gentamicin, metronidazole and cefoxitin) for 2 weeks and APC analysed in the GI tract thereafter. Again it was noted that depletion of the bacterial microbiome abrogated MHC class II expression in the small intestine IEC and prevented upregulation after TBI but had no effect on hematopoietic APC (FIG. 3D E). To further confirm the relationship between the microbiome and MHC class-II expression by IEC, WT mice were rendered dysbiotic by co-housing with IL-17RA^{-/-} mice, as previously described (Varelias et al., 2017). This resulted in increased MHC class-II expression by IEC (FIG. 3F G) and enhanced donor CD4⁺ T cell expansion after BMT (FIG. 3H), consistent with DAMP/PAMP driven response by recipient APC.

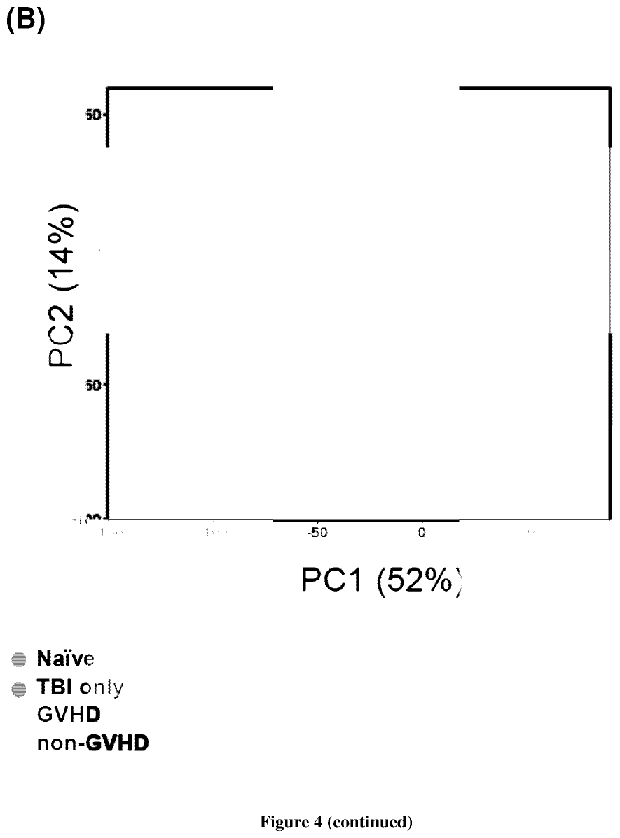

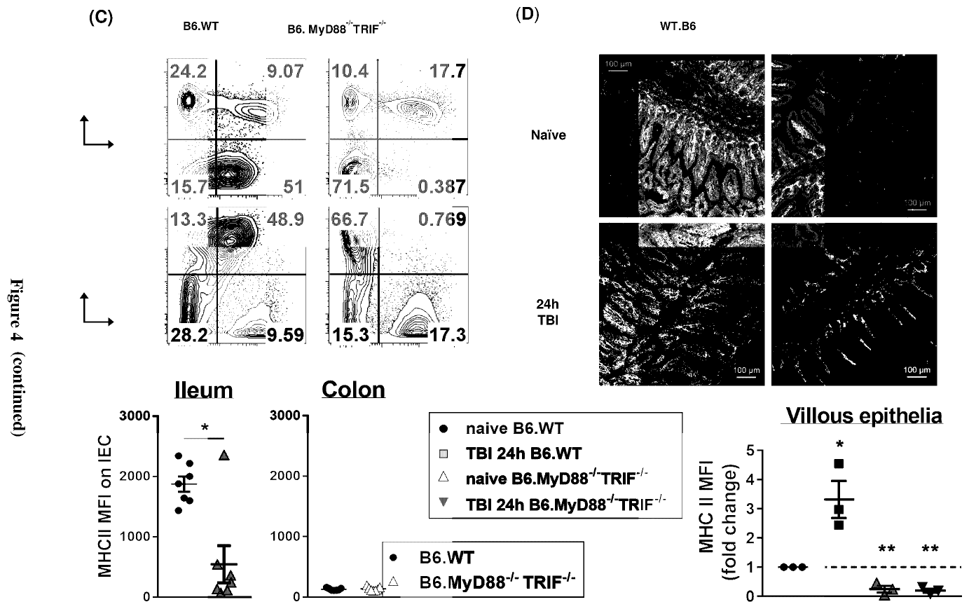

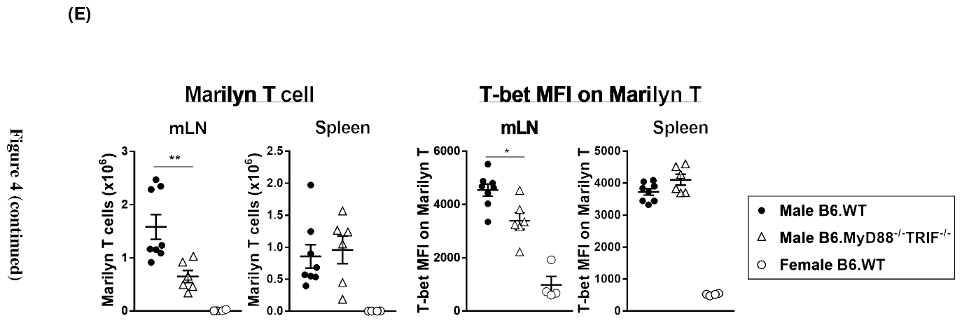

PAMP signalling drives MHC presentation by IEC. The functionality of MHC class-II on IEC was confirmed by examining the ability of these cells to promote activation (CD69 expression) and proliferation (CFSE dilution) of antigen-specific Marilyn CD4 T cells after co-culture (FIG. 4A). Given that IEC from the small intestine express high levels of MHC class-II at steady state, and this is enhanced after TBI and BMT, the molecular mechanisms of antigen presentation in IEC early after BMT were focussed on. RNAseq analysis was performed using sort-purified IEC (CD45^{neg} Villin-YFP⁺) from the small intestine of nave mice, or mice post-TBI only, post-transplant GVHD (with grafts including T cells) and post-transplant non-GVHD (with grafts excluding T cells). Principal component analysis (PCA) demonstrated a strong separation of gene expression profiles between nave mice and all other groups, confirming that the transcriptional landscape was most influenced by TBI (FIG. 4B). Single-sample gene-set enrichment analysis (ssGSEA) was utilized to map pathways that are differentially regulated between the nave and other study groups. This analysis demonstrated that enhanced antigen presentation and processing pathways are equally augmented in mice receiving TBI alone and those receiving T cell replete grafts that developed GVHD (not shown). These pathways involved toll-like receptor (TLR) signalling, including TLR3 and 4, nuclear factor kappa B (NFkB) and mitogen-activated protein (MAP) kinase activation. Given the juxtaposition of IEC and the gut microbiome, and the importance of the latter to GVHD, the relationship between damage/pathogen-associated molecular patterns (DAMP/PAMP) signalling and antigen presentation within MHC class-II by APC in the GI tract was further investigated. The ileum and colon from nave B6 mice (B6.WT) and MyD88, TRIF-deficient mice (B6.MyD88^{-/-}TRIF^{-/-}) that are unable to signal downstream of TLR ligation were compared. Comparable MHC class-II expression was noted in hematopoietic populations (CD45⁺EpCAM^{neg}) while IEC (CD45^{neg} EpCAM⁺) from B6.MyD88^{-/-} TRIF^{-/-} mice completely lacked MHC class-II expression (FIGS. 4C and 4D). In order to confirm that this pathway was relevant for driving donor T cell expansion, HY-specific luciferase expressing donor CD4 T cells were transplanted into male B6.WT or B6.MyD88^{-/-}TRIF^{-/-} recipients. Consistent with the preferential expression of MHC class II in small intestine, T cell expansion was attenuated in that organ but not colon or spleen. Furthermore, alloantigen-specific T cells were preferentially expanded and Th1 differentiated in the draining (mesenteric) lymph node (FIG. 4E).

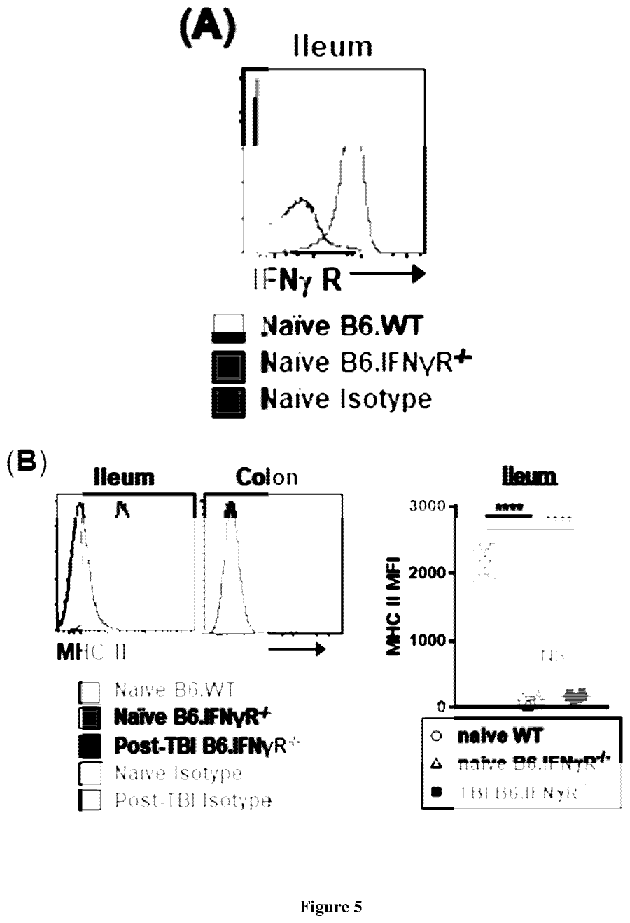

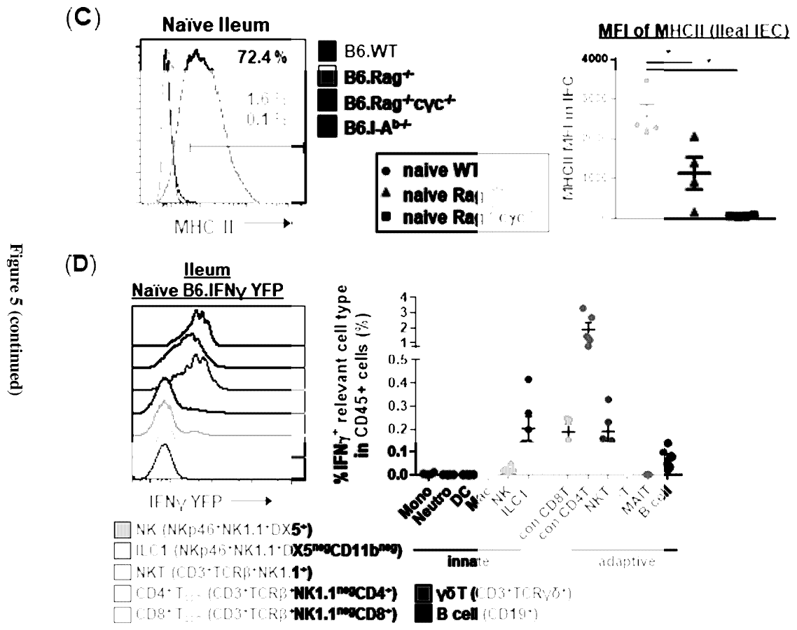

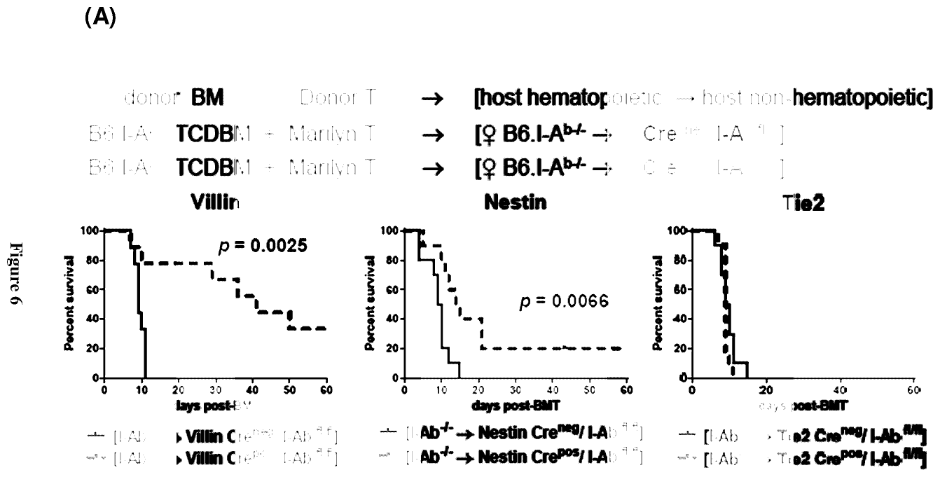

High levels of IFN-γ are secreted by both ILC1 and by conventional T cells in the small intestine. The mechanisms controlling MHC class-II expression by IEC, focusing on interferon (IFN) γ signalling were delineated, since this cytokine is known to potently induce MHC class-II expression. IEC do indeed express the IFN-γ receptor (IFNγR) (FIG. 5A) and B6.IFNγ12^{-/-} mice, like B6.MyD88^{-/-}TRIF^{-/-} mice, did not express MHC class-II at steady-state or after TBI (FIG. 5B). To understand the potential sources of IFNγ driving the expression of MHC class II, Rag^{-/-} common-γ-chain^{-/-} which lack innate lymphocyte populations, and Rag^{-/-} mice that lack only conventional TCR-rearranged T cells, were analyzed. These data confirmed that MHC class II was driven by both innate and conventional T cell populations (FIG. 5C). After TBI-induced inflammation, conventional T cells were required for the upregulation of MHC class II in the ileum. Consistent with this, IFN-γ secretion from innate cells was noted at steady-state predominantly in CD200r⁺ type 1 innate lymphoid cells (ILC1), while CD4 T cells were the predominant source within conventional T cells (FIG. 5D). In contrast, TBI induced IFN-γ secretion from conventional T cells (CD4⁺ T_{con}, CD8⁺ T_{con}) but not innate lymphocyte populations (not shown). Interestingly, the induction of IFN-γ secretion after TBI was only seen in conventional T cells in the GI tract and was completely absent in draining lymph nodes (mLN). Thus, constitutive MHC class-II expression on IEC in the ileum causes local T cell activation and IFNγ secretion, consistent with the induction of a local cytokine feed-forward cascade that amplifies MHC class-II expression on IEC. In order to confirm that the induction of MHC class-II expression by IEC was a direct effect of IFNγ, intestinal organoids were cultured from the small intestine of B6.MHC class-II-GFP or B6.WT mice with IFNγ. EpCAM⁺ epithelial cells in organoids failed to express MHC class-II in standard culture conditions, but did so rapidly in the presence of IFNγ.

IECs are sufficient to induce MHC class-II dependent GVHD. MHC class II antigen presentation by IEC was investigated to determine if it could initiate acute GVHD. It was previously demonstrated that other host non-hematopoietic APC, including cells of mesenchymal origin could press MHC class II. In the present disclosure, three murine lines expressing Cre recombinase (Cre) driven off villin, nestin and Tie2 promoters, which are lineage markers for IEC, mesenchymal and endothelial cells, respectively, were used to define the relevance of MHC class-II expressed by these non-hematopoetic cell populations in the GI tract in driving GVHD. Lineage-restricted expression in the intestine was determined by Cre-driven YFP expression. Villin-expressing cells are epithelial cell adhesion molecule (EpCAM)⁺CD45^{neg} epithelial cells and align on the surface of villi. Nestin-expressing cells are CD45^{neg}Vimentin⁺α-smooth muscle actin (αSMA)+, consistent with mesenchymal cells. CD45^{neg} Tie2-expressing cells are CD31⁺Ter119^{neg} endothelial cells. I-A^{b} is the MHC class-II molecule expressed in B6 mice, and its expression can be deleted in Cre-expressing (Cre^{pos}) cell lineages using a foxed I-A^{b} gene (I-A^{b-fl/fl}).

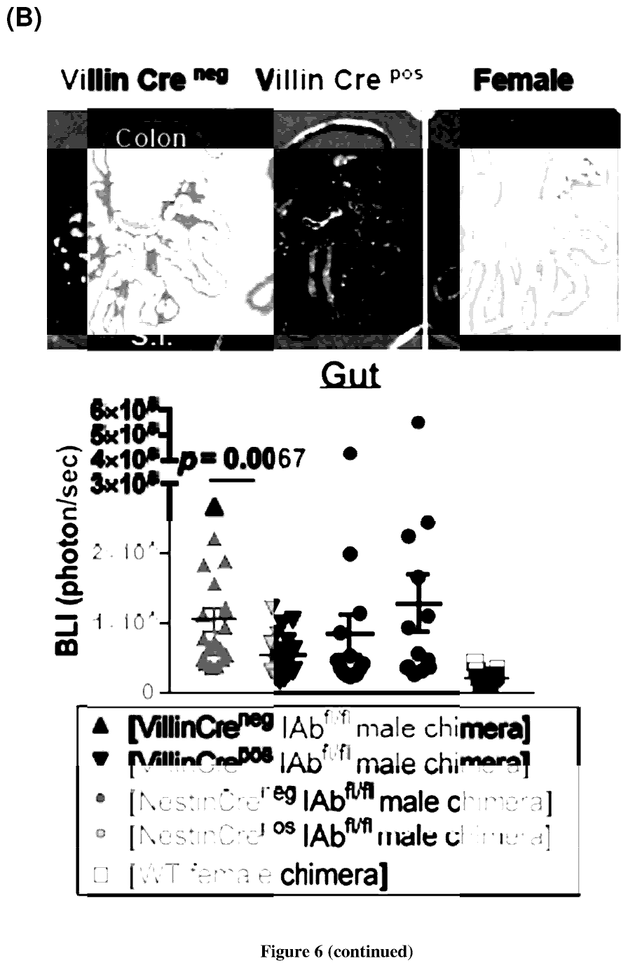

Lethality of GVHD between transplanted mice in which lineage restricted non-hematopoietic cells can (Cre^{neg}) or cannot (Cre^{pos}) present MHC class-II-loaded alloantigen was compared. B6 male nestin, villin or Tie2 Cre^{pos}I-A^{b-fl/fl} mice and relevant Cre^{neg}I-A^{b-fl/fl} mice were reconstituted with female I-A^{b} deficient bone marrow (B6.I-A^{b-/-} BM), thus generating BM chimeras lacking the capacity for antigen (Ag) presentation by hematopoietic APC. Three months later, these BM chimeras were used as BMT recipients and transplanted with female B6.1-A^{b-/-} BM (to reconstitute hematopoiesis whilst preventing Ag presentation by donor APC). Female HY-CD4 transgenic (Tg) T cells (Marilyn T cells), which are specific for I-A^{b} complexed with HY (male) derived peptide were transplanted to induce GVHD. Survival analyses demonstrated a critical role for alloantigen presentation by villin and less so nestin-expressing cells in the initiation of lethal GVHD (FIG. 6A). A significant reduction in luciferase-expressing Marilyn T cell expansion in the gut when recipient villin-expressing cells alone could not present antigen was also demonstrated (FIG. 6B).

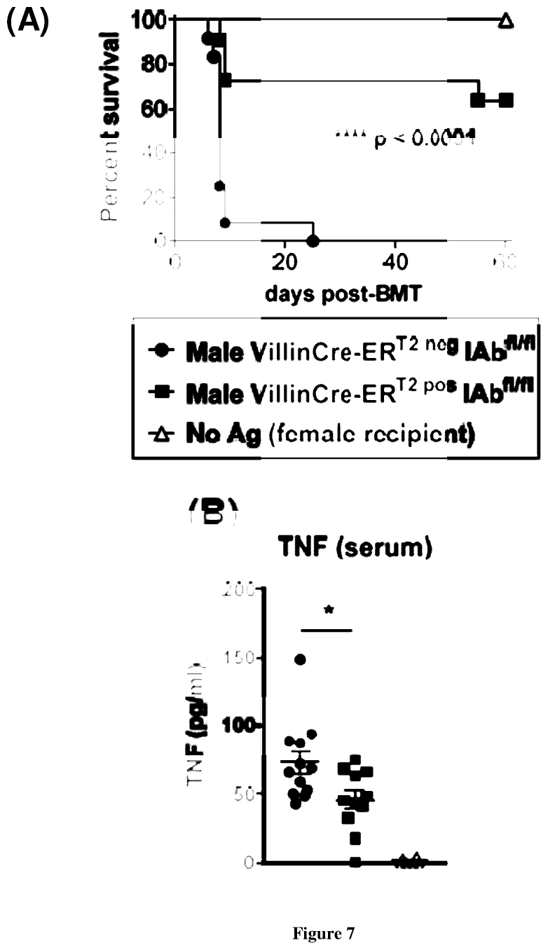

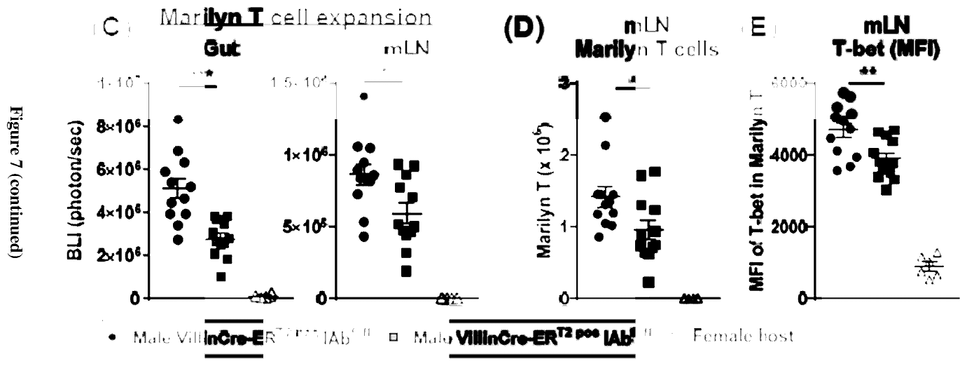

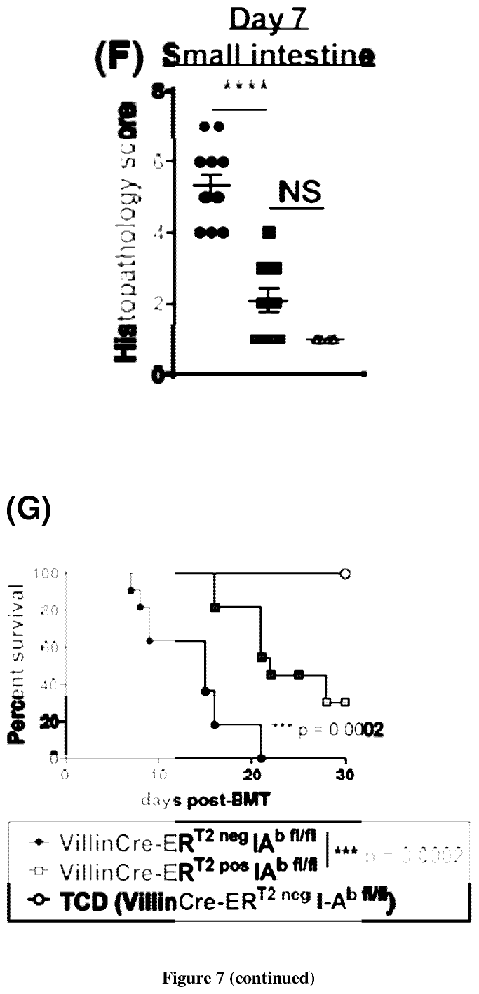

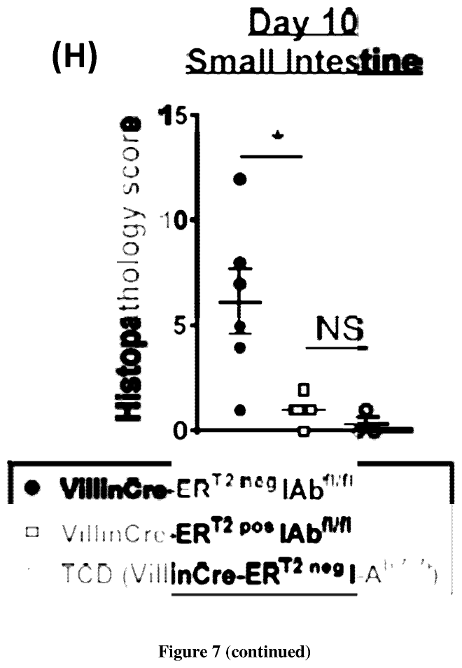

IEC are necessary for induction of MCH class-II dependent GCHD within the GI tract. The critical role of villin-expressing cells in lethal acute GVHD was examined and verified using a second system where recipients express tamoxifen-dependent Cre-recombinase (villinCre-ER^{T2}) under the control of the villin promoter. Donor and host hematopoietic APC are intact but mice have (Cre-ER^{T2-neg}I-A^{b-fl/fl}), or lack (Cre-ER^{T2-pos}I-A^{b-fl/fl}) MHC class-II on IEC after tamoxifen administration. Surprisingly, despite the presence of all other types of APC, the lack of MHC class-II⁺ IEC resulted in profound protection from acute GVHD (FIG. 7A). Consistent with this, we detected reduced levels of TNF in sera (FIG. 7B), decreased alloantigen-reactive HY-CD4 Tg T cell expansion in the gut (mesenteric lymph nodes (mLN)) (FIG. 7C 7D), a reduction in T-bet expression (FIG. 7E), and prevention of GVHD pathology in the GI tract (FIG. 7F). These data establish that eliminating antigen presentation by villin-expressing APC restricts alloreactive T cell expansion and Th1 differentiation in the GI tract and prevents lethal acute GVHD. We corroborated this finding with wild type (WT) polyclonal CD4+ T cells from BALB/c mice (H-2^{d}), transplanted into MHC-mismatched female VillinCre-ER^{T2-neg}I-A^{b-fl/fl} or Cre-ER^{T2-pos}I-A^{b-fl/fl} recipients (H-2^{b}). VillinCre-ER^{T2-pos}I-A^{b-fl/fl} recipients did not develop gut GVHD (FIGS. 7G and 7H).

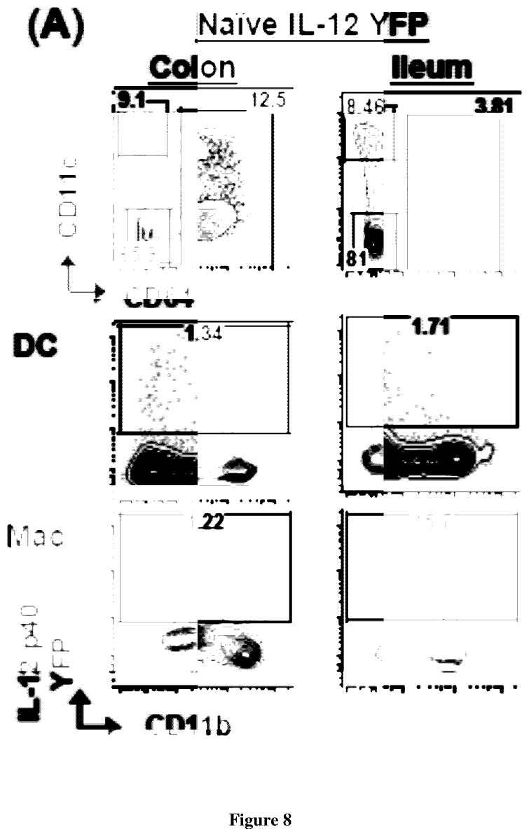

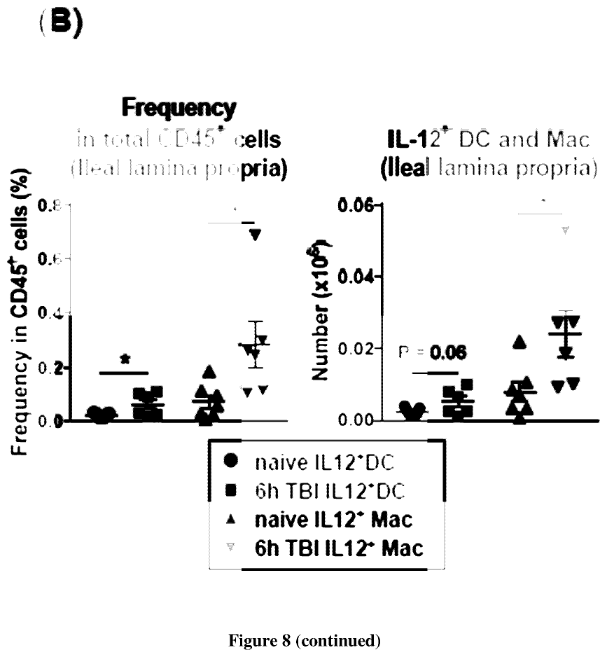

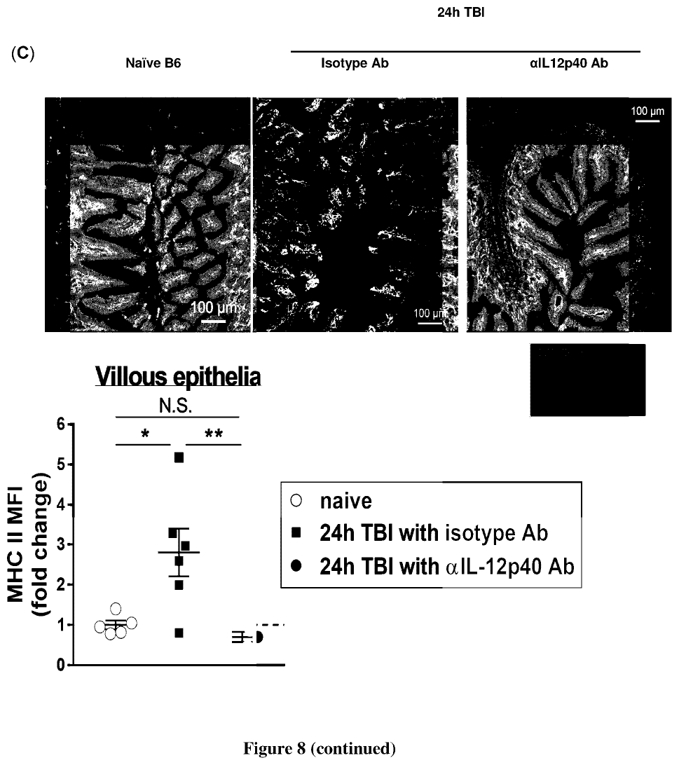

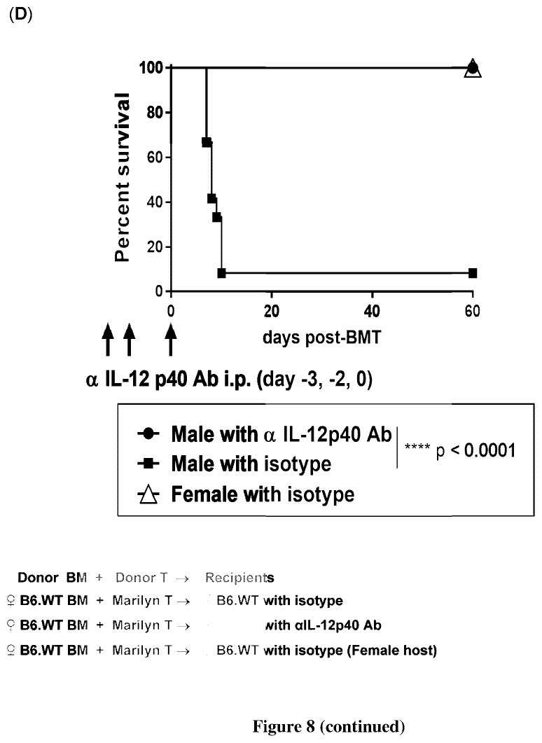

IL-12 neutralization prevents MHC class-II expression by IEC. Finally, the role of IL-12 in the IFNγ dependent induction of MHC class II expression by IEC was explored. It was noted that IL-12p40 was preferentially secreted by macrophages in the ileum but not colon and this was enhanced after TBI (FIG. 8A). IL-12p40 inhibition attenuated IFNγ secretion by ILC1 and CD4 T cells (FIG. 8B). Importantly, the acquisition of APC function by IEC could be prevented by IL-12p40 inhibition prior to TBI (FIG. 8C), and this completely prevented acute GVHD lethality (FIG. 8D). Given that these therapeutic antibodies are immediately available, this provides a strategy to prevent acute GVHD in the clinic, as long as inhibition is initiated before conditioning.

### DISCUSSION

In this study, the pivotal role of IEC in antigen presentation is described, how this is responsible for the initiation of GVHD in the GI tract, and the interplay between the microbiome and IEC that controls this process. The interactions between the microbiome and host immunity are well defined and involve various DAMP/PAMP signalling motifs, including Toll-like Receptors, Nod-like receptors and short-chain fatty acids. However, the ability of the microbiome to shape antigen presentation by gut epithelial cells has not previously been defined. The present disclosure shows that under homeostatic conditions IL-12-secreting macrophages drive IFN-γ secretion by ILC1 in a microbiome and MyD88/TRIF-dependent manner, resulting in MHC class-II expression by IEC in the ileum. Conditioning with TBI invokes additional IL-12 secretion by dendritic cells and rapid IFN-γ secretion by conventional recipient T cells in the GI tract, culminating in rapid and dramatic enhancement of MHC class-II expression by IEC. Critically, deletion of MHC class-II in villin-expressing enterocytes prevents donor T cell priming, differentiation and GVHD in the GI tract after BMT. Thus, the present disclosure describes novel interactions between the microbiome and innate immune cells in the GI tract, which keep IEC continuously poised to respond to antigen challenges. The present disclosure also shows that when this delicate balance is perturbed by inflammatory signals, overt immunopathology ensues.

These results not only identify a new axis of antigen presentation that drives disease in the GI tract, but also define rapidly testable pathways for therapeutic intervention that include microbiome modification, macrophage depletion, broad Toll-like receptor inhibition and IL-12/23 and/or IFNγ inhibition. While it is clear that hematopoietic cells include the principal APC that drive MHC class I-dependent acute GVHD, this does not hold true for MHC class-II-dependent acute GVHD (Koyama et al., 2012). Importantly, while acute GVHD can be mediated by inflammatory cytokines independent of cognate T cell-MHC interactions in target tissue, the present disclosure demonstrates that the initiation phase of lethal class-II-dependent acute GVHD does require a cognate T cell-MHC class-II interaction at the epithelial surface of the small intestine. Thus, the present disclosure has identified a novel and critical role of IEC as the non-hematopoietic cell subset involved in antigen presentation that explains the inability of approaches that delete hematopoietic professional recipient APC to prevent acute GVHD in preclinical systems. It is important to note that many professional APC subsets (e.g. dendritic cells) do potently present alloantigen, but the net effect of this function is activation induced death and/or phagocytosis (e.g. by macrophages) of donor CD4 T cells that paradoxically attenuates GVHD. Thus, approaches to delete recipient DC, macrophages or even B cells are generally deleterious and instead amplify GVHD. In contrast, IEC in the ileum present endogenous alloantigen in a truly pathogenic fashion (since deletion attenuates GVHD) and this process is under strict control by soluble mediators secreted by hematopoietic APC (i.e. IL-12) and innate lymphocytes (i.e. IFNγ) in response to the microbiome. The data disclosed herein suggests that the control of antigen presentation within non-hematopoietic cells is an important component of this interactive network.

The secretion of IFN-γ by conventional recipient T cells within 24 hours of TBI, in the absence of transplantation, was unexpected. Since this does not occur in primary or secondary lymphoid organs, despite significant IL-12 secretion at those sites, it seems that localized, likely tissue resident memory T cells respond to local TCR-dependent signals in the context of IL-12. It is likely that this reflects a response to local pathogen-derived peptide-MHC complexes that are absent in primary lymph nodes and drive a tissue specific feed forward cascade in the GI tract. Indeed, since IL-12 instructs IFN-γ secretion by innate and adaptive lymphocytes, systemic neutralisation of IL-12, commencing before TBI prevents the IFNγ-dependent expression of MHC class-II by IEC that initiates acute GVHD. Thus, IL-12 inhibition starting before conditioning would appear an attractive adjunct approach to prevent GVHD. Intriguingly, the data disclosed herein also suggest that strategies to prevent GVHD based on T cell depletion would be most efficacious when completed prior to the initiation of conditioning.

The crucial role for antigen-presentation by IEC in the ileum as a distinct anatomical site is intriguing, particularly given that the colon is the site where most microbiome-derived DAMP/PAMP signals reside. Despite this, colon epithelial cells do not acquire antigen presentation function, potentially reflective of the extensive mucus layer that acts as an effective barrier at this site, as opposed to the ileum. Although MHC class-II expression by non-hematopoietic cells such as fibroblasts, endothelial cells and epithelial cells has been described, its functional role in antigen presentation has remained ambiguous. Indeed it has been considered to promote tolerance rather than inflammation. In addition, since the role of MHC class-II is classically defined by exogenous antigen presentation following phagocytosis or endocytosis, the relative importance for endogenous antigen presentation within MHC class-II has been less clear. In contrast, the results defined herein definitively demonstrate that IEC present alloantigen to CD4⁺ T cells to initiate GVHD. Furthermore, the data highlight a number of critical pathways that can be modulated prior to transplant to prevent the initiation of acute GVHD in the GI tract, focusing on the microbiome, DAMP/PAMP signalling and the downstream cytokines IL-12 and IFNγ.

### REFERENCES

Adolph et al. (2013) Nature, 503:272-276.
el Marjou et al. (2004) Genesis, 39:186-193.
Hamilton et al. (2012) Am J Gastroenterol, 107(5):761-767.
Hashimoto et al. (2002) Genesis, 32: 152-153.
Hirota et al. (2011) Nature Immunology, 12:255-263.
Koyama et al. (2012) Nat Med, 18:135-142.
Yamamoto et al. (2003) Science, 301:640-643.
Zhang et al. (2017) Sci Immunol 2.

1. A method of reducing the risk of a transplant recipient developing graft versus host disease (GVHD), the method comprising administering a composition to the transplant recipient to modulate the transplant recipient's gut microbiome, and/or administering a composition to reduce MHC class II expression on intestinal epithelial cells (IEC) in the transplant recipient, wherein the composition is administered prior to the transplant recipient receiving pre-transplant conditioning therapy.

2. The method of claim 1, wherein the method comprises administering the composition to modulate the transplant recipient's gut microbiome at least three days prior to the transplant recipient receiving the pre-transplant conditioning therapy, and/or administering the composition to reduce MHC class II expression on IEC in the transplant recipient at least three days prior to the transplant recipient receiving the pre-transplant conditioning therapy.

3. The method of claim 1, wherein the method comprises administering the composition to modulate the transplant recipient's gut microbiome at least seven days prior to the transplant recipient receiving the pre-transplant conditioning therapy, and/or administering the composition to reduce MHC class II expression on IEC in the transplant recipient at least seven days prior to the transplant recipient receiving the pre-transplant conditioning therapy.

4. The method of claim 1, wherein the method comprises administering the composition to modulate the transplant recipient's gut microbiome at least two weeks prior to the transplant recipient receiving the pre-transplant conditioning therapy, and/or administering the composition to reduce MHC class II expression on IEC in the transplant recipient at least two weeks prior to the transplant recipient receiving the pre-transplant conditioning therapy.

5. The method of any one of claims 1 to 4, wherein the composition to modulate the transplant recipient's gut microbiome comprises an antibiotic.

6. The method of claim 5, wherein the antibiotic is a broad-spectrum antibiotic.

7. The method of claim 5, wherein the antibiotic is selected from vancomycin, polymixin B, amphotericin B, gentamycin, cefuroxime, and/or ceftriaxone.

8. The method of any one of claims 1 to 4, wherein the composition to modulate the transplant recipient's gut microbiome comprises a probiotic or fecal transplant.

9. The method of any one of claims 1 to 8, wherein the method comprises first administering an antibiotic to the transplant recipient, and then administering a probiotic and/or fecal transplant to the transplant recipient.

10. The method of any one of claims 1 to 9, wherein the composition to reduce MHC class II expression on IEC in the transplant recipient comprises at least one of an inhibitor of an inflammatory cytokine, an innate defense regulator, Toll-like receptor inhibitor, an antimicrobial peptide, and/or a compound that depletes T-cells.

11. The method of claim 10, wherein the composition to reduce MHC class II expression on IEC in the transplant recipient comprises an inhibitor of an inflammatory cytokine.

12. The method of claim 11, wherein the inhibitor of an inflammatory cytokine reduces activity of an inflammatory cytokine selected from IL-12, IL-23 and/or IFN-γ.

13. The method of claim 12, wherein the inhibitor reduces activity of IL-12 and/or IL-23 in the transplant recipient.

14. The method of any one of claims 10 to 13, wherein the inhibitor of the inflammatory cytokine is an antibody that binds to the inflammatory cytokine and/or reduces binding of the inflammatory cytokine to its receptor.

15. The method of claim 14, wherein the antibody binds to IL-12 and/or IL-23.

16. The method of claim 14 or claim 15, wherein the antibody binds to the p40 subunit of IL-12 and IL-23.

17. The method of claim 16, wherein the antibody is ustekinumab.

18. The method of claim 15, wherein the antibody is risankizumab.

19. The method of any one of claims 10 to 13, wherein the inhibitor of the inflammatory cytokine is a compound that reduces cells within the GI tract that secrete one or more inflammatory cytokines.

20. The method of claim 19, wherein the one or more inflammatory cytokines are selected from IL-12, IL-23 and IFNγ.

21. The method of claim 20, wherein the cells within the GI tract are macrophages that secrete IL-12.

22. The method of claim 20, wherein the cells within the GI tract are innate type-1 lymphocytes (ILC1) that secrete IFNγ.

23. The method of any one of claims 10 to 22, wherein the composition to reduce MHC class II expression on IEC in the transplant recipient comprises an innate defense regulator.

24. The method of claim 23, wherein the innate defense regulator is dusquetide.

25. The method of any one of claims 10 to 20, wherein the composition to reduce MHC class II expression on IEC in the transplant recipient comprises an antimicrobial peptide.

26. The method of any one of claims 10 to 25, wherein the compound that depletes T-cells is selected from an antithymocyte globulin and/or an anti-CD52 antibody.

27. The method of claim 26, wherein the anti-CD52 antibody is alemtuzumab.

28. The method of claim 26 or claim 27, wherein the antithymocyte globulin is selected from thymoglobulin or ATGAM.

29. The method of any one of claims 1 to 28, wherein the method reduces the risk of the transplant recipient developing acute GVHD.

30. A composition that modulates a transplant recipient's gut microbiome, and/or a composition that reduces MHC class II expression on IEC in a transplant recipient, for reducing the risk of the transplant recipient developing GVHD.

31. Use of a composition that modulates a transplant recipient's gut microbiome and/or a composition that reduces MHC class II expression on IEC in a transplant recipient in the manufacture of a medicament for reducing the risk of the transplant recipient developing GVHD.

32. A pharmaceutical combination comprising:
i) a composition that modulates a transplant recipient's gut microbiome; and
ii) a composition that reduces MHC class II expression on IEC in a transplant recipient.
